(19) United States
(12) Patent Application Publication
(10) Pub. No.: US 2021/0102245 A1
(43) Pub. Date: Apr. 8, 2021

(54) DIRECT NUCLEIC ACID ANALYSIS OF ENVIRONMENTAL AND BIOLOGICAL SAMPLES

(71) Applicant: Spartan Bioscience Inc., Ottawa (CA)

(72) Inventors: Chris Harder, Dunrobin (CA); Christine Dobson, Ottawa (CA); Paul Lem, Ottawa (CA); Alan Mears, Ottawa (CA); Jeffrey Do, Ottawa (CA); Ali Khatib, Ottawa (CA)

(21) Appl. No.: 16/608,014

(22) PCT Filed: Apr. 27, 2018
(86) PCT No.: PCT/CA18/50495

§ 371 Date: Oct. 24, 2019

Related U.S. Application Data
(60) Provisional application No. 62/617,158, filed on Jan. 12, 2018.
(60) Provisional application No. 62/538,055, filed on Jul. 28, 2017.
(60) Provisional application No. 62/492,017, filed on Apr. 28, 2017.

(51) Int. Cl. C12Q 1/6844; C12Q 1/6806; C12N 15/10

(52) U.S. Cl. CPC C12Q 1/6844; C12Q 1/6806; C12Q 2521/101; C12Q 2527/143; C12Q 2527/149; C12N 15/1017

(57) ABSTRACT

Methods and apparatuses are described for nucleic acid analysis of environmental water samples and biological samples without the need for purification.

### TECHNICAL FIELD OF THE INVENTION

The invention relates to the field of diagnostic assays, in particular, nucleic acid amplification-based assays for the detection of microorganisms in environmental samples and nucleic acids in biological samples.

### BACKGROUND

Analysis of environmental samples (e.g., water from an industrial cooling tower, untreated fresh water, etc.) and biological samples (e.g., cell samples, body fluid samples, swab samples) by polymerase chain reaction (PCR) based methods is challenging due to the presence of contaminants in the sample that may inhibit the reaction. Dilution of the sample prior to analysis may reduce the concentration of the inhibitor to a level that does not adversely affect the reaction; however, the sensitivity of the analysis may be compromised. Purification of the nucleic acid from a sample may also adversely affect analysis by degrading the nucleic acid, or the purification step may be ineffective in removing all or some of the inhibitory contaminants. There is a need for methods capable of detecting low levels of nucleic acids (e.g., from microorganisms) present in environmental and biological samples which often comprise PCR inhibitors.

### SUMMARY OF THE INVENTION

In one aspect, the present disclosure encompasses the discovery that by concentrating an environmental sample and contacting the concentrated sample with a nucleic acid amplification reagent without any intervening steps (e.g., without extraction or purification of the nucleic acid from the sample), nucleic acids from a microorganism present in the environmental sample, for example a water sample, may be amplified (e.g., by PCR) and detected. The present disclosure also encompasses the insight that use of nucleic acid amplification reagents at concentrations substantially higher than typically used is advantageous when contacting a concentrated sample, or a biological sample, with a nucleic acid amplification reagent without any intervening steps, and performing the reaction. Without wishing to be bound by any particular theory, the present disclosure proposes that use of nucleic acid amplification reagents at concentrations substantially higher than typically used is particularly advantageous for direct amplification of a sample that may include PCR inhibitors and/or a low concentration of nucleic acid.

Accordingly, in one aspect, the disclosure features a method comprising steps of obtaining an environmental sample comprising a microorganism, wherein the microorganism comprises a nucleic acid; concentrating the environmental sample to produce a concentrated sample, wherein the microorganism is concentrated about 2-fold to about 125-fold in the concentrated sample as compared to the environmental sample; contacting the concentrated sample with a nucleic acid amplification reagent in a reaction vessel, wherein the concentrated sample is directly contacted with the nucleic acid amplification reagent without any intervening steps; and performing a nucleic acid amplification reaction on the nucleic acid from the microorganism in the concentrated sample.

The present disclosure also encompasses the discovery that existing methods for detecting and quantifying the levels of certain microorganisms in environmental samples (e.g., by PCR) are inaccurate because they involve significant periods of time (e.g., 1-3 days) between sample collection and analysis. Without wishing to be bound by any particular theory, the present disclosure proposes that growth and/or degradation of the microorganism (e.g., bacteria) in between collection and analysis is a significant contributor to the measurement errors.

Accordingly, in one aspect, the disclosure features a method comprising steps of obtaining an environmental sample from a source, wherein the environmental sample comprises a microorganism and the microorganism comprises a nucleic acid; contacting the environmental sample (optionally a concentrated environmental sample as described above) with a nucleic acid amplification reagent in a reaction vessel, wherein the environmental sample (optionally the concentrated sample) is directly contacted with the nucleic acid amplification reagent without any intervening steps; and performing a nucleic acid amplification reaction on the nucleic acid from the microorganism in the environmental sample (optionally the concentrated sample), wherein the nucleic acid amplification reaction is completed within less than 1 day from when the environmental sample was originally collected from the source. In some embodiments, the amplification reaction is completed within less than 12 hours, less than 10 hours, less than 8 hours, less than 6 hours, less than 4 hours, less than 2 hours, less than 1 hour, less than 45 minutes, less than 30 minutes, less than 15 minutes, less than 10 minutes, less than 5 minutes, or less than 1 minute from when the environmental sample was originally collected from the source.

The present disclosure also encompasses the discovery that existing methods for detecting and quantifying the levels of certain microorganisms in environmental samples (e.g., by PCR) are inadequate because they are not performed with sufficient frequency. Without wishing to be bound by any particular theory, the present disclosure proposes that the speed at which certain microorganisms (e.g., bacteria) can grow is such that testing needs to be performed at higher frequency, particularly when currently used testing methods underestimate the actual levels of certain microorganisms (e.g., bacteria).

Accordingly, in one aspect, the disclosure features a method comprising steps of obtaining an environmental sample comprising a microorganism from a source, wherein the microorganism comprises a nucleic acid; contacting the environmental sample (optionally a concentrated environmental sample) with a nucleic acid amplification reagent in a reaction vessel, wherein the sample (optionally the concentrated sample) is directly contacted with the nucleic acid amplification reagent without any intervening steps; and performing a nucleic acid amplification reaction on the nucleic acid from the microorganism in the sample (optionally the concentrated sample) (optionally within less than 1 day from when the environmental sample was originally collected from the source), and then repeating the method on a new environmental sample from the same source within less than one month (e.g., monthly or on the same day of each consecutive month). In some embodiments, the method is repeated within less than one week (e.g., weekly or on the same day of each consecutive week). In some embodiments, the method is repeated within 24 hours (e.g., on a daily basis). In some embodiments, the method is repeated within 12 hours (e.g., twice a day).

In some embodiments, an environmental sample is a water sample collected from a source selected from the group consisting of industrial cooling tower water, untreated fresh water, waste water, stagnant water, wash water, grey water and water obtained from a lavatory, shower, bathtub, toilet, sink.

In some embodiments, a microorganism is a bacteria, cyanobacteria, virus, protozoa, fungus or rotifer. In some embodiments, the bacteria is selected from the group consisting of Alicyclobacillus, Aeromonas, Bacteroides, Bifidobacterium, Campylobacter, Citrobacter, Clostridia, Enterobacter, Enteroccocus, Escherichia, Eubacterium, Klebsiella, Lactobacillus, Legionella, Listeria, Mycobacterium, Pseudomonas, Raoultella, Salmonella, Shigella, Streptococcus, Vibrio and combinations thereof. In some embodiments, a bacteria is selected from the group consisting of Legionella pneumophila, Legionella longbeachae, Legionella bozemannii, Legionella micdadei, Legionella feeleii, Legionella dumoffii, Legionella wasdworthii, Legionella anisa and combinations thereof. In some embodiments, a bacteria is Escherichia coli.

In some embodiments, an environmental sample may be concentrated to produce the concentrated sample by filtration, evaporation and/or centrifugation. In some embodiments, an environmental sample may be concentrated to produce the concentrated sample by filtration. In some embodiments, a filtration step comprises washing a retentate and/or eluting the concentrated sample from the filter. In some embodiments, filtration is performed using a hydrophilic filter membrane. In some embodiments, filtration is performed using a hydrophilic polyethersulfone (PES) filter membrane.

In some embodiments, a nucleic acid amplification reaction comprises a DNA polymerase at a concentration of at least 1.0 U/reaction and a primer at a concentration of at least 0.2 μM. In some embodiments, a reaction volume is 20 μL. In some embodiments, the nucleic acid amplification reaction comprises a probe at a concentration ranging from about 1.0 μM to about 14 μM. In some embodiments, a DNA polymerase is at a concentration ranging from about 3.4 U/reaction to about 45 U/reaction. In some embodiments, a primer is at a concentration ranging from about 1.3 μM to about 15 μM. In some embodiments, a nucleic acid amplification reaction comprises a DNA polymerase at a concentration ranging from at least 12 U/reaction to about 21 U/reaction, a primer at a concentration ranging from at least 4.0 μM to about 7.0 μM and a probe at a concentration ranging from at least 3.5 μM to about 7.0 μM.

In some embodiments, the method further comprises a step of determining whether an amplification product was produced as a result of the nucleic acid amplification reaction. In some embodiments, a nucleic acid amplification reagent does not comprise a reagent which is designed to resist DNA polymerase inhibitors.

In some embodiments, the method does not include a step of lysing the microorganism. In some embodiments, the method does not include a further step of purifying the nucleic acid from the microorganism. In some embodiments, the method further comprises a step of determining whether an amplification product was produced as a result of the nucleic acid amplification reaction.

In one aspect, the disclosure features a method comprising steps of obtaining a sample comprising a nucleic acid, contacting the sample with a nucleic acid amplification reagent in a reaction vessel, wherein the sample is directly contacted with the nucleic acid amplification reagent without any intervening steps and wherein the nucleic acid amplification reagent comprises a DNA polymerase at a concentration ranging from at least 6 U/reaction to about 42 U/reaction, a primer at a concentration ranging from at least 2.0 μM to about 14 μM and a probe at a concentration ranging from at least 1.9 μM to about 14 μM; and performing a nucleic acid amplification reaction on the nucleic acid from the sample.

In some embodiments, a sample is selected from the group consisting of an environmental sample and a biological sample. In some embodiments, an environmental sample is a concentrated sample. In some embodiments, an environmental sample is a water sample selected from the group consisting of industrial cooling tower water, untreated fresh water, waste water, stagnant water, wash water, grey water and water obtained from a lavatory, shower, bathtub, toilet, sink.

In some embodiments, an environmental sample comprises a microorganism and wherein the microorganism comprises a nucleic acid. In some embodiments, a microorganism is a bacteria, cyanobacteria, virus, protozoa, fungus or rotifer. In some embodiments, a bacteria is selected from the group consisting of Alicyclobacillus, Aeromonas, Bacteroides, Bifidobacterium, Campylobacter, Citrobacter, Clostridia, Enterobacter, Enteroccocus, Escherichia, Eubacterium, Klebsiella, Lactobacillus, Legionella, Listeria, Mycobacterium, Pseudomonas, Raoultella, Salmonella, Shigella, Streptococcus, Vibrio and combinations thereof. In some embodiments, a bacteria is selected from the group consisting of Legionella pneumophila, Legionella longbeachae, Legionella bozemannii, Legionella micdadei, Legionella feeleii, Legionella dumoffii, Legionella wasdworthii, Legionella anisa and combinations thereof. In some embodiments, a bacteria is Escherichia coli.

In some embodiments, a biological sample is selected from the group consisting of a cell sample, a body fluid sample and a swab sample. In some embodiments, a biological sample is collected from a foodstuff or a mammal. In some embodiments, a mammal is a human.

In some embodiments, the method further comprises a step of determining whether an amplification product was produced as a result of the nucleic acid amplification reaction. In some embodiments, a step of obtaining comprises collecting a swab sample.

### BRIEF DESCRIPTION OF FIGURES

FIG. 1 depicts exemplary results demonstrating detection of Legionella pneumophilia genomic DNA by PCR in concentrated environmental samples using increasing amounts of dNTPs, polymerase, primers and probe.

FIG. 2 depicts exemplary data collected and analyzed during a study.

FIG. 3 depicts exemplary method of calculating time to action.

FIG. 4 depicts exemplary results for Spartan qPCR v. laboratory qPCR for spiked water samples after a 24-hour delay.

FIG. 5A depicts exemplary direct culture plate of water sample. FIG. 5B depicts exemplary colony PCR results.

FIG. 6 depicts exemplary growth of L. pneumophilia in a water sample from cooling tower O11.

FIG. 7 depicts annotated results from weeks 1-7 of the study.

FIG. 8 depicts annotated results from weeks 8-14 of the study.

### DEFINITIONS

As used herein the following terms shall have the meanings indicated, unless indicated otherwise:

As used herein, the term “about” when used in reference to a numerical value, means plus or minus 10%.

As used herein, the terms “amplification” or “amplify” refer to methods known in the art for copying a target sequence from a template nucleic acid, thereby increasing the number of copies of the target sequence in a sample. Amplification may be exponential or linear. A template nucleic acid may be either DNA or RNA. The target sequences amplified in this manner form an “amplified region” or “amplicon.” While the exemplary methods described hereinafter relate to amplification using PCR, numerous other methods are known in the art for amplification of target nucleic acid sequences (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al. (1990). Eds. Academic Press, San Diego, Calif. pp 13-20; Wharam et al. (2001). Nucleic Acids Res. 29(11): E54-E54; Hafner et al. (2001). Biotechniques. 30(4): 852-6, 858, 860 passim. Further amplification methods suitable for use with the present methods include, for example, reverse transcription PCR (RT-PCR), ligase chain reaction (LCR), transcription-based amplification system (TAS), nucleic acid sequence based amplification (NASBA) reaction, self-sustained sequence replication (3SR), strand displacement amplification (SDA) reaction, boomerang DNA amplification (BDA), Q-beta replication, isothermal nucleic acid sequence based amplification or real-time PCR.

As used herein, the term “bacterial growth” or “growth” refers to a test result impacted by bacterial growth if the test value is at least 2-fold higher for a sample tested after a time delay (e.g., shipping delay of 1-3 days) as compared to a sample tested in parallel without a time delay.

As used herein, the term “bacterial degradation” or “degradation” refers to a test result impacted by bacterial degradation if the test value is at least 2-fold lower for a sample tested after a time delay (e.g., shipping delay of 1-3 days) as compared to a sample tested in parallel without a time delay.

As used herein, the term “biological sample” refers to a sample obtained from a biological source. In some embodiments, a biological sample is a body fluid sample (e.g., blood, cerebrospinal fluid, saliva, urine) or a cell sample. In some embodiments, a biological sample is a swab sample. In some embodiments, the biological sample is collected from a foodstuff or a mammal. In some embodiments, the mammal is a human.

As used herein, the term “colony forming units/milliliter” (CFU/mL) refers to a unit of measurement for estimating the number of bacterial cells grown on a bacterial plate.

As used herein, the term “direct qPCR” refers to methods comprising addition of a non-concentrated environmental sample directly into a qPCR system. Direct qPCR differs from Spartan qPCR and laboratory qPCR in that the environmental sample is not concentrated (e.g., by filtration) before analysis. In some embodiments, a LOD of direct qPCR is greater than 200 GU/mL. In some embodiments, a LOD of Spartan qPCR is less than 10 GU/mL. In some embodiments, a LOD of laboratory qPCR is less than 10 GU/mL.

As used herein, the term “DNA” refers to some or all of the DNA from a microorganism (e.g., bacteria, cyanobacteria, virus, protozoa, fungus, rotifer) or from the nucleus of a cell. DNA may be intact or fragmented (e.g., physically fragmented or digested with restriction endonucleases by methods known in the art). In some embodiments, DNA may include sequences from all or a portion of a single gene or from multiple genes. In some embodiments, DNA may be in the form of a plasmid. In some embodiments, DNA may be linear or circular. In some embodiments, DNA may include sequences from one or more chromosomes, or sequences from all chromosomes of a cell.

As used herein, the term “environmental sample” refers to a sample obtained from a non-biological source. In some embodiments, an environmental sample is an aqueous sample, e.g., a water sample. In some embodiments, a water sample is obtained from an industrial, health-care or residential facility or setting. In some embodiments, a water sample is obtained from a natural setting (e.g., lake, stream, pond, reservoir or other water source). In some embodiments, an environmental sample is a water sample obtained from an industrial cooling tower. In some embodiments, an environmental sample is a water sample obtained from an untreated fresh water source. In some embodiments, an environmental sample is a waste water sample. In some embodiments, an environmental sample is standing water (e.g., stagnant water), wash water or grey water. In some embodiments, an environmental sample is a water sample obtained from a lavatory, shower, bathtub, toilet or sink.

As used herein, the term “forward primer” refers to a primer that hybridizes to the anti-sense strand of dsDNA. A “reverse primer” hybridizes to the sense-strand of dsDNA.

As used herein, the term “genomic units/milliliter” (GU/mL) refers to a unit of measurement for estimating the number of DNA copies (e.g., bacterial DNA copies) present in a sample. In some embodiments, GU/mL refers to “genomic equivalents/mL” or “GE/mL”.

As used herein, the terms “hybridize” and “hybridization” refer to a process where two complementary or partially-complementary nucleic acid strands anneal to each other as a result of Watson-Crick base pairing. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementarities will form stable hybrids, while those having lower complementarities will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

As used herein, the term “laboratory culture” or “culture,” refers to the process of adding a sample to a nutrient-rich plate and allowing bacteria to grown in individual spots (colonies). In some embodiments, colonies are counted to determine the number of bacteria in a given sample (expressed as CFU/mL). Culture often involves pre-treatment of a sample to remove non-Legionella bacteria and antibiotic-treated culture plates to prevent growth of non-Legionella bacteria. In some embodiments, laboratory culture results are available by 10-14 days.

As used herein, the term “laboratory qPCR” refers to a method of concentrating bacteria, isolating their DNA, and quantifying the amount of DNA using qPCR. In some embodiments, laboratory qPCR is performed in accordance with ISO standard 12869:2012 “Water quality—Detection and quantification of Legionella ssp. and/or Legionella pneumophilia by concentration and genic amplification by quantitative polymerase chain reaction (qPCR).”

As used herein, the term “Legionella pneumophilia” (L. pneumophilia) refers to a species of Legionella bacteria and is the primary causative agent of Legionnaires' disease. In some embodiments, there are 15 subtypes of L. pneumophilia that can be detected by methods described herein.

As used herein, the term “limit of detection” (LOD) refers to the lowest quantity of L. pneumophilia that is distinguishable from the absence of L. pneumophilia within the confidence limits of a method.

As used herein, the term “microorganism” refers to a microscopic organism that may be single-celled or multicellular. Examples of microorganisms include bacteria, cyanobacteria, viruses, protozoa, fungus and rotifers. In some embodiments, a bacterium is of the genus Alicyclobacillus, Aeromonas, Bacteroides, Bifidobacterium, Campylobacter, Citrobacter, Clostridia, Enterobacter, Enteroccocus, Escherichia, Eubacterium, Klebsiella, Lactobacillus, Legionella, Listeria, Mycobacterium, Pseudomonas, Raoultella, Salmonella, Shigella, Streptococcus, Vibrio or a combination thereof. In some embodiments, the Legionella species is Legionella pneumophila, Legionella longbeachae, Legionella bozemannii, Legionella micdadei, Legionella feeleii, Legionella dumoffii, Legionella wasdworthii or Legionella anisa. In some embodiments, the Escherichia species is Escherichia coli.

As used herein, the term “nucleic acid” refers broadly to DNA, segments of a chromosome, segments or portions of DNA, cDNA, and/or RNA. Nucleic acids may be derived or obtained from an originally isolated nucleic acid sample from any source (e.g., isolated from, purified from, amplified from, cloned from, reverse transcribed from sample DNA or RNA). In some embodiments, the source of a nucleic acid may be a bacteria, cyanobacteria, virus, protozoa, fungus or rotifer. Nucleic acids include those resident in an environmental sample, preferably a water sample. In some embodiments, the source of the nucleic acid may be a biological sample, for example, a body fluid sample, a cell sample or a swab sample.

As used herein, the term “negative” refers to a test result, or group of test results, that comprise an undetectable level of L. pneumophilia, such as, a result below the LOD of the test.

As used herein, the term “positive” refers to a test result, or group of test results that comprise detectable levels of L. pneumophilia at or above the LOD of the test.

As used herein, the term “quantitative polymerase chain reaction” (qPCR) refers to a technology for amplifying sections of DNA. In some embodiments, quantitative PCR amplifies DNA and quantifies the amount of DNA. As used herein, the term “sense strand” refers to the strand of double-stranded DNA (dsDNA) that includes at least a portion of a coding sequence of a functional protein. “Anti-sense strand” refers to the strand of ds DNA that is the reverse complement of the sense strand.

As used herein, the term “Spartan qPCR” is performed using methods described herein. In some embodiments, a method described herein is Spartan Legionella Detection System. In some embodiments, Spartan qPCR is completed within 2 hours, 1 hour, 45 minutes, 30 minutes or 15 minutes after collection of the sample from a source (e.g,, an environmental source). In some embodiments, Spartan qPCR quantifies the amount of L. pneumophilia bacterial DNA (GU/mL) in a water sample (e.g., from an industrial cooling tower system).

As used herein, the term “swab sample” means a sample obtained with a collection tool. The collection tool may include a small piece of cotton or soft porous foam on the end of the tool, but is not required to. In general, a swab sample may be collected by contacting a sample source with a physical structure. Any physical structure that collects a swab sample when contacted with the sample source may be used for this purpose. In some embodiments, the physical structure may comprise an absorbent material (e.g., cotton). In some embodiments, the physical structure may be made of plastic and may collect the swab sample as a result of friction.

In some embodiments, a swab sample is collected from a mammal (e.g., a human, dog, cat, cow, sheep, pig, etc.). In some embodiments, a mammal is a human. In some embodiments, a swab sample is collected from an open body cavity (e.g., mouth, nose, throat, ear, rectum, vagina, and wound). In some embodiments, a swab sample is a buccal sample. In some embodiments, a buccal sample may be collected by contacting (e.g., touching and/or swiping) the inside of a cheek. In some embodiments, a buccal sample may be collected by contacting with a tongue rather than a cheek. In some embodiments, a swab sample is collected from a body surface (e.g., skin). In some embodiments, a swab sample is collected from the palm of a hand, inside the folds of the pinna of an ear, an armpit, or inside a nasal cavity.

In some embodiments, a swab sample is collected from a foodstuff. In some embodiments, a foodstuff is raw. In some embodiments, a foodstuff is a fruit, a vegetable, a meat, a fish, or a shellfish. In some embodiments, meat is pork, beef, chicken or lamb. In some embodiments, a swab sample may be collected by touching and/or swiping the relevant foodstuff.

In some embodiments, the term “without any intervening steps” refers to directly contacting the nucleic acid amplification reagent with sample. For example, a concentrated sample comprising, for example, whole bacteria, cyanobacteria, virus, protozoa, fungus or rotifer. In some embodiments, a sample is a biological sample. In some embodiments, the term “without any intervening steps” comprises performing a method without steps such as lysing microorganisms present in a concentrated sample and/or purifying nucleic acids from microorganisms present in a concentrated sample. In some embodiments, the term “without any intervening steps” comprises performing a method without steps such as extracting or purifying nucleic acids present in a biological sample. Directly contacting may be achieved by, for example, placing the nucleic acid amplification reagent in a reaction vessel, then bringing the nucleic acid amplification reagent into contact with a sample (e.g., a concentrated environmental sample, a biological sample) by, for example, flicking the reaction vessel, inverting the reaction vessel, shaking the reaction vessel, vortexing the reaction vessel, etc.

### Description

Nucleic acids are routinely analyzed for clinical diagnosis, prognosis and treatment of diseases and conditions such as heritable genetic disorders, infections due to pathogens and cancer. Generally the sample type analyzed is a biological sample such as a cell sample, body fluid sample or swab sample. Nucleic acid analysis is also performed for detection of contaminating pathogens in environmental samples such as industrial water samples. Commonly used analysis methods include a step of extracting or purifying the nucleic acid from the sample prior to amplification. However, this step takes additional time, often requires use of expensive and/or special reagents and can result in loss or degradation of the nucleic acid. Therefore, methods that do not require extraction or purification of the nucleic acid prior to performing amplification (e.g., directly contacting the sample with the nucleic acid amplification reagent) are advantageous. Challenges to overcome when using methods that directly analyze a sample include the presence of PCR inhibitors in the sample and/or low concentration of nucleic acid. The present application describes methods of detecting nucleic acids which include concentrating a sample prior to contact with nucleic acid amplification reagent and/or use of nucleic acid amplification reagent at concentrations that are substantially higher than typically used in amplification reactions.

This application describes, inter alia, methods of detecting nucleic acids from a microorganism present in an environmental sample (e.g., an aqueous sample, e.g., water sample) by concentrating the environmental sample to produce a concentrated sample, such that the microorganisms are concentrated as compared to the environmental sample, and contacting the concentrated sample, without any intervening steps, with a nucleic acid amplification reagent and performing a nucleic acid amplification reaction. In some embodiments, the method does not include a step of lysing the microorganism. In some embodiments, the method does not include a step of purifying the nucleic acid from the microorganism. In some embodiments, the method uses a nucleic acid amplification reagent at concentrations that are substantially higher than typically used in amplification reactions.

This application also describes methods of detecting nucleic acids present in other types of samples, such as biological samples (e.g., cell sample, body fluid sample, swab sample) by contacting a sample with a nucleic acid amplification reagent without any intervening steps. In some embodiments, the method uses a nucleic acid amplification reagent at concentrations that are substantially higher than typically used in amplification reactions.

Real-time PCR-based methods have been successfully applied to Legionella monitoring of hot sanitary water (which can be described as “clean water”). However, PCR-based testing and monitoring of “dirty water” samples, that may also comprise various organic and inorganic contaminants (e.g., from industrial cooling tower systems, untreated freshwater), for microorganisms has proven challenging. The contaminants found in these water sources are often inhibitors of nucleic acid polymerases. Attempts to extract or purify the nucleic acid from the samples prior to amplification have had mixed success. In some instances, the nucleic acid is degraded or otherwise lost from the sample, or the inhibitors are inefficiently removed.

The effects of PCR inhibitors co-extracted with DNA from industrial cooling tower water systems can be mitigated by further dilution of the sample. However, this may result in a decreased sensitivity of the method, especially when the abundance of Legionella in the water is low, leading to false-negative results (Baudart et al., J App Micro (2015) 118(5):1238-1249).

Purification or extraction of DNA from the sample may also mitigate the effects of PCR inhibitors. Diaz-Flores et al. performed quantitative PCR on 65 water samples collected from cooling towers, sanitary water, nebulizer and spa matrices (BMC Microbiol (2015) 15:91). Prior to PCR the samples were treated with a lysis buffer, vortexed, incubated at 95° C. and vortexed again to collect the DNA. However, even with this level of purification, 8 of 65 samples (12.3%) demonstrated partial or complete inhibition of PCR.

For reasons such as this, it is recommended that environmental water samples be subjected to DNA purification techniques prior to performing PCR. For example, ISO/Technical Specification 12869:2012 suggests that extraction of DNA by lysing microorganisms purifies the DNA and eliminates PCR inhibitors. Suggested extraction methods include physical (e.g., cycles of freezing and thawing), chemical (e.g. guanidine thiocyanate buffer) or biological (e.g., enzyme digestion) methods.

The requirement for DNA purification prior to performing PCR introduces a time-consuming, labor-intensive, and costly step in the process. For example, the GeneDisc® Rapid Microbiology System (Pall Corp.) for Legionella quantitative PCR (qPCR) requires a GeneDisc® DNA Extractor (a 165-pound instrument that performs ultrasound, boiling, and DNA capture using purification columns) and a GeneDisc® Cycler (a 33-pound instrument that performs qPCR on the purified DNA sample) to perform the method.

Researchers have attempted to perform PCR directly on lysed and diluted environmental water samples; however this has resulted in a high rate of PCR inhibition. For example, Miyamoto et al. analyzed water collected from 49 cooling towers using a semi-nested PCR method to detect Legionella species (Miyamoto et al., Appl. Environ. Microbiol. (1997) 63(7): 2489-2494). Following lysis and purification of the DNA by protease K and detergent treatment, 30% of the samples contained PCR inhibitors. Of the samples containing PCR inhibitors, 6 were successfully amplified only in the second round of PCR, likely as result of the further dilution of inhibitors.

Even when DNA is extracted from environmental water samples, there is still an appreciable PCR inhibition rate. For example, PCR inhibition was observed in 2.7% of DNA samples extracted from water collected from 37 cooling towers following concentration and filtration of the water and purification of the DNA using a High Pure PCR template preparation kit (Roche Diagnostics) (Joly et al., Appl. Environ. Microbiol. 7 (2006) 2(4): 2801-2808). In another study, PCR inhibition was observed in 5% of DNA samples extracted from water collected from cooling water towers for detection of Legionella (Ng et al., Lett. Appl. Microbiol. (1997) 24(3):214-16).

Legionella may also be quantified by culture methods, however contamination may not be detected, or underestimated, in some samples. The CDC conducted proficiency testing of 20 culture laboratories and found that Legionella concentrations in water samples were underestimated by an average of 1.25 logs or 17-fold (Lucas et al., Water Res. (2011) 45:4428-4436). Also, culture testing incorrectly reported water samples as negative for Legionella an average of 11.5% of the time when in fact they were positive. Furthermore, standard procedures for recovery of Legionella, including shipping, filtration, and heat/acid enrichment, are known to lead to a significant loss of cell culturability (Boulanger and Edelstein, J. Appl. Microbiol. (1995) 114:1725-1733; McCoy et al. Water Res. (2012) 46:3497-3506; Roberts et al., Appl. Environ. Microbiol. (1987) 53:2704-2707). Furthermore, culture testing is logistically disadvantageous as it requires shipment of samples to a central laboratory and 10-14 days for Legionella growth.

A sensitive method for performing a nucleic acid amplification reaction on nucleic acids from a microorganism in a concentrated environmental sample, and which does not require any intervening steps prior to contacting the concentrated sample with a nucleic acid amplification reagent, would be advantageous.

The present disclosure also encompasses the discovery that existing methods for detecting and quantifying the levels of certain microorganisms in environmental samples (e.g., by PCR) are innacurate because they involve significant periods of time (e.g., 1-3 days) between sample collection and analysis. Without wishing to be bound by any particular theory, the present disclosure proposes that growth and/or degradation of the microorganism (e.g., bacteria) in between collection and analysis is a significant contributor to the measurement errors.

Accordingly, in one aspect, the disclosure features a method comprising steps of obtaining an environmental sample from a source, wherein the environmental sample comprises a microorganism and the microorganism comprises a nucleic acid; contacting the environmental sample (optionally a concentrated environmental sample as described above) with a nucleic acid amplification reagent in a reaction vessel, wherein the environmental sample (optionally the concentrated sample) is directly contacted with the nucleic acid amplification reagent without any intervening steps; and performing a nucleic acid amplification reaction on the nucleic acid from the microorganism in the environmental sample (optionally the concentrated sample), wherein the nucleic acid amplification reaction is completed within less than 1 day from when the environmental sample was originally collected from the source. In some embodiments, the amplification reaction is completed within less than 12 hours, less than 10 hours, less than 8 hours, less than 6 hours, less than 4 hours, less than 2 hours, less than 1 hour, less than 45 minutes, less than 30 minutes, less than 15 minutes, less than 10 minutes, less than 5 minutes, or less than 1 minute from when the environmental sample was originally collected from the source.

The present disclosure also encompasses the discovery that existing methods for detecting and quantifying the levels of certain microorganisms in environmental samples (e.g., by PCR) are inadequate because they are not performed with sufficient frequency. Without wishing to be bound by any particular theory, the present disclosure proposes that the speed at which certain microorganisms (e.g., bacteria) can grow is such that testing needs to be performed at higher frequency, particularly when currently used testing methods underestimate the actual levels of certain microorganisms (e.g., bacteria).

Accordingly, in one aspect, the disclosure features a method comprising steps of obtaining an environmental sample comprising a microorganism from a source, wherein the microorganism comprises a nucleic acid; contacting the environmental sample (optionally a concentrated environmental sample) with a nucleic acid amplification reagent in a reaction vessel, wherein the sample (optionally the concentrated sample) is directly contacted with the nucleic acid amplification reagent without any intervening steps; and performing a nucleic acid amplification reaction on the nucleic acid from the microorganism in the sample (optionally the concentrated sample) (optionally within less than 1 day from when the environmental sample was originally collected from the source), and then repeating the method on a new environmental sample from the same source within less than one month (e.g., monthly or on the same day of each consecutive month). In some embodiments, the method is repeated within less than one week (e.g., weekly or on the same day of each consecutive week). In some embodiments, the method is repeated within 24 hours (e.g., on a daily basis). In some embodiments, the method is repeated within 12 hours (e.g., twice a day).

### Concentration of Microorganisms

As detailed herein, a sample, which may be an environmental sample, is collected and microorganisms present in the sample are concentrated. Concentration of the microorganisms present in the sample comprises removal and/or reduction of an aqueous component of the sample to produce a “concentrated sample.” In some embodiments, a concentrated sample comprises an increased concentration, level, percentage and/or amount of microorganism as compared to the environmental sample.

Concentration of a microorganisms in a sample may be performed without lysis of the microorganism. Concentration of a microorganism in a sample may be performed without release, extraction and/or purification of the nucleic acid from the microorganism.

In some embodiments, a sample may be concentrated by filtration, for example using a filter membrane. In some embodiments, a filter membrane is hydrophilic. In some embodiments, a filter membrane is a hydrophilic polyethersulfone (PES) filter. In some embodiments, filtration comprises a step of washing a retentate and/or eluting a concentrated sample from the filter. In some embodiments, washing is performed using a buffer comprising water, 1X GoTaq colorless buffer (Promega, Cat. No. M7921), 2.5 mM magnesium chloride, 0.1% w/v sodium azide, and 0.05% w/v sodium hexametaphosphate. In some embodiments, a wash buffer is phosphate buffered saline. A volume of wash buffer used to wash a retentate may vary depending upon the amount environmental sample that is filtered. In some embodiments about 1 mL, about 2 mL, about 3 mL, about 4 mL, about 5 mL, about 6 mL, about 7 mL, about 8 mL, about 9 mL, about 10 mL or more of wash buffer is used. In some embodiments, a volume of wash buffer is 2 mL. A washing step may be performed one or more times.

In some embodiments, a concentrated sample may be eluted from a filter membrane. Elution of a concentrated sample may be performed using a buffer that is the same, or similar to a wash buffer. For example, an elution buffer may comprise water, 1X GoTaq colorless buffer (Promega, Cat. No. M7921), 2.5 mM magnesium chloride, 0.1% w/v sodium azide, and 0.05% w/v sodium hexametaphosphate. In some embodiments, an elution buffer is phosphate buffered saline. A volume of elution buffer used to elute a retentate from a filter may vary depending on the degree of concentration to be achieved. In some embodiments, a volume of elution buffer is about 100 μL, about 200 μL, about 300 μL, about 400 μL, about 500 μL about 600 μL, about 700 μL, about 800 μL, about 900 μL about 1 mL, about 2 mL, about 5 mL or more. An elution buffer may be contacted with a filter membrane one or more times. For example, an elution buffer may be pulsed back and forth across a membrane multiple times in order to elute a retentate and produce a concentrated sample. In some embodiments, an elution buffer is pulsed back and forth across a membrane about 5, about 10, about 15, about 20, about 25, about 50 times or more to elute a retentate and produce a concentrated sample. In some embodiments, an elution buffer is pulsed back and forth across a membrane about 20 times.

In some embodiments, an environmental sample is concentrated by evaporation and/or centrifugation.

In some embodiments, a sample is concentrated about 0.5- fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 50-fold, 60-fold, 70-fold, 80-fold, 90-fold, 100-fold, 125-fold, 150-fold, 175-fold, 200-fold, 300-fold, 400-fold, 500-fold, 600-fold or ranges within as compared to an environmental sample. In some embodiments, a sample is concentrated about 500-fold as compared to an environmental sample. In some embodiments, a sample is concentrated about 375-fold as compared to an environmental sample. In some embodiments, a sample is concentrated about 250-fold as compared to an environmental sample. In some embodiments, a sample is concentrated about 125-fold as compared to an environmental sample. In some embodiments, a sample is concentrated about 63-fold as compared to an environmental sample. In some embodiments, a sample is concentrated about 31-fold as compared to an environmental sample. In some embodiments, a sample is concentrated about 16-fold as compared to an environmental sample. In some embodiments, a sample is concentrated about 8-fold as compared to an environmental sample. In some embodiments, a sample is concentrated about 0.5-fold as compared to an environmental sample.

In some embodiments, an environmental sample may be concentrated within a range. For example, from about 0.5-fold to about 500-fold as compared to an environmental sample. In some embodiments, a sample may be concentrated by about 8-fold to about 375-fold as compared to an environmental sample. In some embodiments, a sample may be concentrated by about 16-fold to about 250-fold as compared to an environmental sample. In some embodiments, a sample may be concentrated by about 31-fold to about 125-fold as compared to an environmental sample. In some embodiments, a sample may be concentrated by about 16-fold to about 31-fold as compared to an environmental sample. In some embodiments, a sample may be concentrated by about 8-fold to about 63-fold as compared to an environmental sample. In some embodiments, a sample may be concentrated by about 2-fold to about 125-fold as compared to an environmental sample.

In some embodiments, microorganisms present in an environmental sample may be lysed prior to concentration of the sample. In some embodiments, lysis may be performed using a surfactant (e.g., an anionic surfactant, an ionic surfactant). In some embodiments, a surfactant is an anionic surfactant (e.g., SDS). In some embodiments, a surfactant concentration in an amplification reaction is less than or equal to about 0.005% (w/v). In some embodiments, lysis may be performed using thermal treatment (e.g., high heat).

A concentrated sample may be directly contacted with a nucleic acid amplification reagent in a reaction vessel without any intervening steps. In some embodiments, the nucleic acid amplification reagent is directly contacted with a concentrated sample comprising, for example, whole bacteria, cyanobacteria, virus, protozoa, fungus or rotifer. In some embodiments, a method without any intervening steps is performed without steps such as lysing microorganisms present in a concentrated sample and/or purifying nucleic acids from microorganisms present in a concentrated sample. Directly contacting may be achieved by, for example, placing a nucleic acid amplification reagent in a reaction vessel, then bringing the nucleic acid amplification reagent into contact with the concentrated sample (e.g., by flicking the reaction vessel, inverting the reaction vessel, shaking the reaction vessel, vortexing the reaction vessel, etc.).

### Amplification of Nucleic Acids

In various embodiments, template nucleic acids from the sample may be amplified using polymerase chain reaction (PCR) or reverse transcription PCR (RT-PCR); however, as noted previously, the skilled artisan will understand that numerous methods are known in the art for amplification of nucleic acids, and that these methods may be used either in place of, or together with, PCR or RT-PCR. For example, without limitation, other amplification methods employ ligase chain reaction (LCR), transcription-based amplification system (TAS), nucleic acid sequence based amplification (NASBA) reaction, self-sustained sequence replication (3SR), strand displacement amplification (SDA) reaction, boomerang DNA amplification (BDA), Q-beta replication, isothermal nucleic acid sequence based amplification, etc. In general, nucleic acid amplification methods, such as PCR, RT-PCR, isothermal methods, rolling circle methods, etc., are well known to the skilled artisan. See, e.g., Saiki, “Amplification of Genomic DNA” in PCR Protocols, Innis et al. (1990). Eds. Academic Press, San Diego, Calif. pp 13-20; Wharam et al. (2001). Nucleic Acids Res. 29(11): E54-E54; Hafner et al. (2001). Biotechniques. 30(4): 852-6, 858, 860 passim.

The nucleic acid amplification reagents that are involved in each of these amplification methods (e.g., enzymes, primers, probes, buffers, surfactants etc.) may vary but are also well known in the art and readily available from commercial sources (e.g., see catalogues from Invitrogen, Biotools, New England Biolabs, Bio-Rad, QIAGEN, Sigma-Aldrich, Agilent Technologies, R&D Systems, etc.). It will also be appreciated that the specific primers and/or probes that are used in any given method will depend on the template nucleic acid and the target sequence that is being amplified and that those skilled in the art may readily design and make suitable primers and/or probes for different template nucleic acids and target sequences. Primers and probes may also be prepared by commercial suppliers (e.g., Integrated DNA Technologies).

In certain embodiments, a nucleic acid amplification reaction of the methods described herein may contain DNA polymerase at a concentration substantially higher than typically used in amplification reactions (e.g., 1.0 U/20 μL reaction). In the embodiments discloses herein, the reaction volume is typically 20 μL. Those skilled in the art, reading the present specification, will appreciate that when the reaction volume is larger or smaller than 20 μL, the amount of DNA polymerase used in the reaction is adjusted accordingly. In some embodiments, a DNA polymerase concentration is at least 1.0 U/reaction, e.g., at least 1.2 U/reaction, at least 1.4 U/reaction, at least 1.6 U/reaction, at least 1.8 U/reaction, at least 2.0 U/reaction, at least 2.2 U/reaction, at least 2.4 U/reaction, at least 2.6 U/reaction, at least 2.8 U/reaction, at least 3.0 U/reaction, at least 3.2 U/reaction, at least 3.4 U/reaction, at least 3.6 U/reaction, at least 3.8 U/reaction, at least 4.0 U/reaction, at least 5.0 U/reaction, at least 6.0 U/reaction, at least 7.0 U/reaction, at least 8.0 U/reaction, at least 9.0 U/reaction, at least 10 U/reaction, at least 11 U/reaction, at least 12 U/reaction, at least 13 U/reaction, at least 14 U/reaction, at least 15 U/reaction, at least 20 U/reaction, at least 25 U/reaction, at least 30 U/reaction, at least 25 U/reaction, at least 30 U/reaction, at least 35 U/reaction, at least 40 U/reaction, at least 45 U/reaction, at least 50 U/reaction or higher. In certain embodiments, a DNA polymerase concentration is 3.4 U/reaction. In some embodiments, a DNA polymerase concentration is 6 U/reaction. In some embodiments, a DNA polymerase concentration is 12 U/reaction. In some embodiments, a DNA polymerase concentration is 21 U/reaction. In some embodiments, a DNA polymerase concentration is 42 U/reaction. In some embodiments, a DNA polymerase concentration ranges from at least 3.4 U/reaction to about 45 U/reaction. In some embodiments, a DNA polymerase concentration ranges from at least 12 U/reaction to about 21 U/reaction. In some embodiments, a DNA polymerase concentration ranges from at least 6 U/reaction to about 42 U/reaction.

In some embodiments, a nucleic acid amplification reaction may contain primer concentrations substantially higher than typically used in amplification reactions (e.g., 0.1-0.2 μM). In some embodiments, a primer concentration in an amplification reaction is at least 0.1 μM, e.g., at least 0.2 μM, at least 0.4 μM, at least 0.6 μM, at least 0.8 μM, at least 1.0 μM, at least 1.2 μM, at least 1.4 μM, at least 1.6 μM, at least 1.8 μM, at least 2.0 μM, at least 2.5 μM, at least 3.0 μM, at least 3.5 μM, at least 4.0 μM, at least 4.5 μM, at least 5.0 μM, at least 5.5 μM, at least 6.0 μM, at least 6.5 μM, at least 7.0 μM, at least 7.5 μM, at least 8.0 μM, at least 8.5 μM, at least 9.0 μM, at least 9.5 μM, at least 10 μM, at least 11 μM, at least 12 μM, at least 13 μM, at least 14 μM, at least 15 μM or higher. In some embodiments, a primer concentration in an amplification reaction is at least 1.3 μM. In some embodiments, a primer concentration in an amplification reaction is at least 2.0 μM. In some embodiments, a primer concentration in an amplification reaction is at least 4.0 μM. In some embodiments, a primer concentration in an amplification reaction is at least 7.0 μM. In some embodiments, a primer concentration in an amplification reaction is at least 14 μM. In some embodiments, a primer concentration in an amplification reaction ranges from at least 1.3 μM to about 15 μM. In some embodiments, a primer concentration in an amplification reaction ranges from at least 4 μM to about 7 μM. In some embodiments, a primer concentration in an amplification reaction ranges from at least 2 μM to about 14 μM. It is to be understood that these values refer to the concentration of each primer (e.g., the concentration of the forward primer or the reverse primer) used in the reaction. In some embodiments, a forward primer concentration in an amplification reaction is 1.3 μM. In some embodiments, a reverse primer concentration in an amplification reaction is 1.3 μM.

In some embodiments, a nucleic acid amplification reaction may contain probe concentrations substantially higher than typically used in amplification reactions (e.g., 0.1-0.2 μM). In some embodiments, a probe concentration in a nucleic acid amplification reaction is at least 0.2 μM, e.g., at least 0.3 μM, at least 0.4 μM, at least 0.5 μM, at least 0.6 μM, at least 0.7 μM, at least 0.8 μM, at least 0.9 μM, at least 1.0 μM, at least 1.2 μM, at least 1.4 μM, at least 1.5 μM, at least 1.6 μM, at least 1.8 μM, at least 2.0 μM, at least 3.0 μM, at least 4.0 μM, at least 5.0 μM, at least 6.0 μM, at least 7.0 μM, at least 8.0 μM, at least 9.0 μM, at least 10 μM, at least 11 μM, at least 12 μM, at least 13 μM, at least 14 μM, at least 15 μM or higher. In some embodiments, a probe concentration in an amplification reaction is at least 1.0 μM. In some embodiments, a probe concentration in an amplification reaction is at least 1.95 μM. In some embodiments, a probe concentration in an amplification reaction is at least 3.9 μM. In some embodiments, a probe concentration in an amplification reaction is at least 6.8 μM. In some embodiments, a probe concentration in an amplification reaction is at least 13.7 μM. In some embodiments, a probe concentration ranges from at least 1.0 μM to about 14 μM. In some embodiments, a probe concentration ranges from at least 3.5 μM to about 7.0 μM. In some embodiments, a probe concentration ranges from at least 1.9 μM to about 14 μM. It is to be understood that these values refer to the concentration of each probe (e.g., a concentration of a mutant probe or a wild-type probe) in an amplification reaction.

In some embodiments, a nucleic acid amplification reaction may contain deoxynucleotides (dNTP) concentrations substantially higher than typically used in amplification reactions (e.g., 0.1-0.2 mM). In some embodiments, a dNTP concentration in a nucleic acid amplification reaction is at least 0.2 mM, e.g., at least 0.3 mM, at least 0.4 mM, at least 0.5 mM, at least 0.6 mM, at least 0.7 mM, at least 0.8 mM, at least 0.9 mM, at least 1.0 mM, at least 1.2 mM, at least 1.4 mM, at least 1.6 mM, at least 1.8 mM, at least 2.0 mM, at least 2.2 mM, at least 2.4 mM, at least 2.6 mM, at least 2.8 mM, at least 3.0 mM or higher. In some embodiments, a dNTP concentration in an amplification reaction is at least 0.3 mM. In some embodiments, a dNTP concentration in an amplification reaction is at least 0.6 mM. In some embodiments, a dNTP concentration in an amplification reaction is at least 1.05 mM. In some embodiments, a dNTP concentration in an amplification reaction is at least 2.1 mM.

In some embodiments, a primer concentration in a nucleic acid amplification reaction is at least 0.5 μM and a probe concentration is at least 0.7 μM. In some embodiments, an amplification reaction comprises a forward primer at a concentration of 1.3 μM, a reverse primer at a concentration of 1.3 μM and a probe at a concentration of 1 μM.

In some embodiments, a nucleic acid amplification reaction contains DNA polymerase, primer, and probe concentrations substantially higher than typically used in amplification reactions. In some embodiments, an amplification reaction comprises a DNA polymerase concentration of 3.4 U/reaction, a primer concentration of 1.3 μM and a probe concentration of 1.0 μM.

In some embodiments, an amplification reaction comprises a DNA polymerase concentration ranging from at least 3.4 U/reaction to about 45 U/reaction, a primer concentration ranging from at least 1.3 μM to about 15 μM and a probe concentration ranging from at least 1.0 μM to about 14 μM. In some embodiments, an amplification reaction comprises a DNA polymerase concentration ranging from at least 12 U/reaction to about 21 U/reaction, a primer concentration ranging from at least 4 μM to about 7 μM and a probe concentration ranging from at least 3.5 μM to about 7 μM. In some embodiments, an amplification reaction comprises a DNA polymerase concentration ranging from at least 6 U/reaction to about 42 U/reaction, a primer concentration ranging from at least 2 μM to about 14 μM and a probe concentration ranging from at least 1.9 μM to about 14 μM.

In some embodiments, a nucleic acid amplification reaction comprises a surfactant (e.g., an anionic surfactant, an ionic surfactant). In some embodiments, a surfactant is an anionic surfactant (e.g., SDS). In some embodiments, a surfactant concentration in an amplification reaction is less than or equal to about 0.005% (w/v). In some embodiments, microorganisms present in a concentrated sample may be lysed following contact with a nucleic acid amplification reagent and heating.

PCR is a technique for making many copies of a specific target sequence within a template DNA. The reaction consists of multiple amplification cycles and is initiated using a pair of primer oligonucleotides that hybridize to the 5′ and 3′ ends of the target sequence. The amplification cycle includes an initial denaturation and typically up to 50 cycles of hybridization, strand elongation (or extension), and strand separation (denaturation). The hybridization and extension steps may be combined into a single step. In each cycle of the reaction, the target sequence between the primers is copied. Primers may hybridize to the copied DNA amplicons as well as the original template DNA, so the total number of copies increases exponentially with time/PCR cycle number. In some embodiments, PCR may be performed according to methods described in Whelan et al. (J. Clin. Microbiol (1995) 33(3):556-561). Briefly, the nucleic acid amplification reagents (PCR reaction mixture) include two specific primers per target sequence, dNTPs, a DNA polymerase (e.g., Taq polymerase), and a buffer (e.g., 1X PCR Buffer. The amplification reaction itself is performed using a thermal cycler. Cycling parameters may be varied, depending on, for example, the melting temperatures of the primers or the length of the target sequence(s) to be extended. As mentioned previously, the skilled artisan is capable of designing and preparing primers that are appropriate for amplifying a target sequence. The length of the amplification primers for use in the present methods depends on several factors including the level of nucleotide sequence identity between the primers and complementary regions of the template nucleic acid and also the temperature at which the primers are hybridized to the template nucleic acid. The considerations necessary to determine a preferred length for an amplification primer of a particular sequence identity are well-known to a person of ordinary skill in the art and include considerations described herein. For example, the length and sequence of a primer may relate to its desired hybridization specificity or selectivity.

In certain embodiments, an environmental sample (optionally a concentrated sample) is contacted with a nucleic acid amplification reagent right after collection of the sample, for example, within about 1-30 minutes of collection. In some embodiments, an environmental sample (optionally a concentrated sample) is contacted with a nucleic acid amplification reagent within about 1 to 60 minutes, within about 1 hour to 8 hours, within about 8 hours to 24 hours, within about 1 day to 3 days, or within about 5 days of collection.

In certain embodiments, a nucleic acid amplification reaction is performed within 120 minutes of contacting an environmental sample (optionally a concentrated sample) with a nucleic acid amplification reagent. In some embodiments, the nucleic acid amplification reaction is performed even sooner, e.g., within 60, 30, 15, 10, 5 or even 1 minute(s) of contacting a concentrated sample with the nucleic acid amplification reagent.

In certain embodiments, a nucleic acid amplification reaction is completed within 120 minutes of contacting a concentrated sample with a nucleic acid amplification reagent. In some embodiments, the nucleic acid amplification reaction is completed even sooner, e.g., within 60, 30, 15, 10, 5 or even 1 minute(s) of contacting a concentrated sample with the nucleic acid amplification reagent.

In certain embodiments, a nucleic acid amplification reaction comprises an initial heat denaturation step of 15 minutes or less. In some embodiments, an initial heat denaturation step is shorter, e.g., 5 minutes or less, 3 minutes or less, 1 minute or less or 30 seconds or less. In some embodiments, an initial heat denaturation is 4.5 minutes. In certain embodiments, an initial heat denaturation step is performed at a temperature in the range of about 85 ° C. to about 105° C., e.g., about 93° C. to about 97° C., about 93° C. to about 95° C., or about 95° C. to about 97° C., etc. In some embodiments, an initial heat denaturation step is performed at about 95° C. In some embodiments, an initial heat denaturation step is performed at about 99° C. In some embodiments an initial heat denaturation step is performed at about 99° C. to about 101° C. In some embodiments, an initial heat denaturation step is performed at about 101° C. to about 103° C.

In some embodiments, an initial heat denaturation step is performed at more than one temperature, for example, at a first temperature followed by a second temperature. In some embodiments, a first temperature is in the range of about 85° C. to about 105° C., e.g., about 93° C. to about 97° C., about 93° C. to about 95° C., or about 95° C. to about 97° C., etc. In some embodiments a second temperature is in the range of about 85° C. to about 105° C., e.g., about 93° C. to about 97° C., about 93° C. to about 95° C., or about 95° C. to about 97° C., etc. In some embodiments, the initial heat denaturation step comprises a first temperature of about 98° C. to about 100° C. for about 30 seconds and a second temperature of about 101° C. to about 103° C. for about 4.5 minutes.

### Detection of Nucleic Acids

The presence of amplified target sequences or amplicons may be detected by any of a variety of well-known methods. For example, in some embodiments electrophoresis may be used (e.g., gel electrophoresis or capillary electrophoresis). Amplicons may also be subjected to differential methods of detection, for example, methods that involve the selective detection of variant sequences (e.g., detection of single nucleotide polymorphisms or SNPs using sequence specific probes). In some embodiments, amplicons are detected by real-time PCR.

Increased endpoint fluorescence above baseline noise levels enable result calling by real-time PCR, though a significant increase in fluorescence is important for accurate quantification. Inhibition of PCR due to inhibitors present in a sample leads to lower fluorescence and inaccurate threshold (Ct) determination when using quantitative PCR threshold analysis methods (Guescini et al. BMC Bioinformatics (2008) 9:326).

Real-time PCR or end-point PCR may be performed using probes in combination with a suitable amplification/analyzer such as the Spartan DX-12 desktop DNA analyzer, or the Spartan Cube which are low-throughput PCR systems with fluorescent detection capabilities. Briefly, probes specific for the amplified target sequence (e.g. molecular beacons, TaqMan probes) are included in the PCR amplification reaction. For example, molecular beacons contain a loop region complementary to the target sequence of interest and two self-complementary stem sequences at the 5′ and 3′ end. This configuration enables molecular beacon probes to form hairpin structures in the absence of a target complementary to the loop. A reporter dye is positioned at the 5′ end and a quencher dye at the 3′ end. When the probes are in the hairpin configuration, the fluorophore and quencher are positioned in close proximity and contact quenching occurs. During PCR, the fluorescently labeled probes hybridize to their respective target sequences; the hairpin structure is lost, resulting in separation of the fluorophore and quencher and generation of a fluorescent signal. In another example, TaqMan probes contain a reporter dye at the 5′ end and a quencher dye at the 3′ end. During PCR, the fluorescent labeled TaqMan probes hybridize to their respective target sequences; the 5′ nuclease activity of the DNA polymerase (e.g., Taq polymerase) cleaves the reporter dye from the probe and a fluorescent signal is generated. When probes that hybridize to different target sequences are used, these are typically conjugated with a different fluorescent reporter dye. In this way, more than one target sequence may be assayed for in the same reaction vessel. The increase in fluorescence signal is detected only if the target sequence is complementary to the probe and is amplified during PCR. A mismatch between probe and target sequences greatly reduces the efficiency of probe hybridization and cleavage.

### EXAMPLES

### Example 1: Detection of Legionella pneumophilia in Water Samples from Cooling Towers

Water samples were collected from 13 different cooling towers. The samples were spiked with Legionella pneumophila (serogroup 1) bacteria (ATCC, Cat. No. 33152) to a final concentration of 3 Genomic Units (GU)/mL. A distilled water sample was also spiked to a final concentration of 3 GU/mL and served as a positive control for amplification of L. pneumophila DNA.

300 mL of each spiked water sample was concentrated using a 0.45 μm pore size hydrophilic polyethersulfone (PES) filter membrane (EMD Millipore, Cat. No. SLHP033RB). The filtered sample was washed by pushing 2 mL of wash buffer across the filter using a 3 mL syringe (VWR, Cat. No. BD309657). The wash buffer was composed of water, 1X GoTaq colorless buffer (Promega, Cat. No. M7921), 2.5 mM magnesium chloride, 0.1% w/v sodium azide, and 0.05% w/v sodium hexametaphosphate. The washed sample was eluted off the filter by pulsing 200 μL of elution buffer back and forth 20 times across the filter using a 1 mL syringe (Covidien Monoject, Cat. No. 1188100777). The composition of the elution buffer was the same as that of the wash buffer. The total sample volume eluted off the filter was 165 μL.

It was empirically determined that the L. pneumophila bacteria in the 165 μL of eluted sample had been concentrated by 500X (because only a minority of the bacteria were eluted off the filter). From this 500X concentrated sample, serial dilutions were performed using elution buffer. This resulted in the following concentrated samples: about 375X, 250X, 125X, 63X, 31X, 16X, 8X, and 0.5X.

Seventeen μL of each concentrated sample (about 500X, 375X, 250X, 125X, 63X, 31X, 16X, 8X, and 0.5X) was added to Spartan Cube reaction cartridges (Spartan Bioscience Inc.). Three μL of PCR master mix was pipetted into each reaction cartridge so that the final concentrations were: 1.3 μM of forward and reverse primers (Forward sequence: 5′-TTGTCTTATAGCATTGGTGCCG-3′ (SEQ ID NO:1) and Reverse sequence: 5′-CCAATTGAGCGCCACTCATAG-3′ (SEQ ID NO:2)), 1.0 μM probes (Sequence: 5′-Cal Fluor610-CAATTGAGCGCCACTCATAG-BHQ-2-3′ (SEQ ID NO:3)), 200 μM dNTPs (Promega, Cat. No. U1330) and 0.17 Units/μL of Hot Start Taq Polymerase (Promega, Cat. No. D6101), i.e., 3.4 Units total of Hot Start Taq Polymerase per 20 μL reaction.

The reaction cartridges were inserted into Spartan Cube devices (Spartan Bioscience Inc.) and the following thermal cycling program was performed:

| | | |
|---|---|---|
| Temperature | Dwell time | Count |

| | | | | |
|---|---|---|---|---|
| 102.5° | C. | 5 | sec | 1 |
| 99° | C. | 270 | sec | 1 |
| 102.5° | C. | 5 | sec | 49 |
| 62° | C. | 15 | sec | 49 |

Samples were determined to be positive when the GU/tube was greater than 0.54 and the fluorescence rise was greater than 750 arbitrary fluorescence units. Each reaction was performed in triplicate. Table 1 shows the quantification of L. pneumophilia DNA using the Spartan Cube device. A result of 0 indicated a negative result due to either 1) insufficient nucleic acid template or 2) inhibition of the DNA polymerase by inhibitors present in the sample. The results demonstrate a higher frequency of negative results from highly concentrated samples due to the presence of inhibitors. Similarly, very dilute samples are below the limit of detection of the assay, and may also result in negative results.

Overall, the results demonstrate that the optimal concentration range for direct PCR with no purification from cooling water samples was about 16X to about 31X. Some samples concentrated about 8X, 63X or 125X were amplified successfully.

TABLE 1
L. pneumophila PCR quantification results (GU/tube)
at different concentrations

| Con- | | | | | | | | | |
|---|---|---|---|---|---|---|---|---|---|
| centration | | | | | | | | | |
| factor | 500X | 375X | 250X | 125X | 63X | 31X | 16X | 8X | 0.5X |
| Distilled | 19.5 | 10.8 | 13.5 | 4.5 | 7.8 | 5.7 | 1.2 | 0.9 | 0 |
| water | | | | | | | | | |
| Sample 1 | 0 | 0 | 0 | 0 | 6.9 | 0.6 | 5.1 | 6 | 0 |
| Sample 2 | 14.7 | 3.3 | 3.3 | 6.9 | 4.5 | 3.9 | 4.8 | 0 | 0 |
| Sample 3 | 63.3 | 61.2 | 38.4 | 23.7 | 9.9 | 7.5 | 1.2 | 0.9 | 0 |
| Sample 4 | 0 | 2.1 | 0.3 | 6.3 | 0 | 0.6 | 4.5 | 0.9 | 0 |
| Sample 5 | 18 | 14.7 | 11.1 | 6.9 | 2.4 | 4.8 | 3 | 1.2 | 0 |
| Sample 6 | 0 | 0 | 0 | 0 | 0 | 6 | 4.5 | 1.5 | 0 |
| Sample 7 | 1.5 | 9 | 15.3 | 14.4 | 10.2 | 9.3 | 1.8 | 2.4 | 0 |
| Sample 8 | 0 | 0 | 0 | 0 | 6.3 | 9.9 | 0.9 | 0 | 0 |
| Sample 9 | 0 | 0 | 0 | 0 | 12 | 14.1 | 2.1 | 1.8 | 0 |
| Sample 10 | 0 | 11.1 | 78.3 | 24.3 | 6 | 15.6 | 6.9 | 0 | 0 |
| Sample 11 | 0 | 0 | 10.5 | 3.9 | 1.8 | 3 | 1.5 | 2.1 | 0 |
| Sample 12 | 0 | 0 | 0 | 1.5 | 9.6 | 5.1 | 2.7 | 4.8 | 0 |
| Sample 13 | 0 | 0 | 0.6 | 13.2 | 0 | 3 | 12 | 5.1 | 0 |

### Example 2: Detection of Legionella pneumophilia in Water Samples

Water samples were collected from four different cooling towers at four different locations in Ottawa, Canada on the same day. The water samples were verified to have undetectable levels of Legionella bacteria using a quantitative PCR (qPCR) assay.

Following this verification, 200 mL of each water sample were poured into a 500 mL plastic bottle and allowed to sit undisturbed for 30 minutes, including a 200 mL control sample of tap water. Next, 110 mL of each water sample were decanted and concentrated using a 0.45 μm polyethersulfone 33-mm filter disk (EMD Millipore, Cat. No. SLHP033RB) and a syringe pump (ThermoFisher Scientific, Cat. No. 8881114030). The filter was washed with 20-30 mL of distilled water and pulsed back and forth with 100 μL 10 times. A final eluent was extracted in two 100 μL fractions of the concentrated sample. The 100 μL fractions were pooled to create a 200 μL eluate. The 200 μL eluate was diluted with water so that the concentration factor was 180X.

5 μL from each the five concentrated samples were added to reaction cartridges (Spartan Bioscience) containing four different PCR master mix final concentrations as described in Table 2. The final reaction volume in each cartridge was 20 μL. The final concentration factor of eluate was 45X (i.e., 180X concentration factor diluted by 5 μL of eluate in 20 μL of final reaction volume).

TABLE 2

| Reagent | 1X | | 2X | | 3.5X | | 7X | |
|---|---|---|---|---|---|---|---|---|
| 5X Colorless GoTaq ® Reaction Buffer | 1X | | 1X | | 1X | | 1X | |
| (Promega, Cat. No. M792B) | | | | | | | | |
| dNTPs (Enzymatics, Cat. No. N2050L) | 0.3 | mM | 0.6 | mM | 1.05 | mM | 2.1 | mM |
| Magnesium chloride (Sigma-Aldrich, | 2.5 | mM | 2.5 | mM | 2.5 | mM | 2.5 | mM |
| Cat. No. M1028) | | | | | | | | |
| GoTaq ® MDx Hot Start Polymerase | 6 | Units | 12 | Units | 21 | Units | 42 | Units |
| (Promega, Cat. No. D6001) | | | | | | | | |
| Forward primer | 2 | μM | 4 | μM | 7 | μM | 14 | μM |
| Reverse primer | 2 | μM | 4 | μM | 7 | μM | 14 | μM |
| Probe | 1.95 | μM | 3.9 | μM | 6.825 | μM | 13.65 | μM |
| Legionella pneumophila genomic DNA | 25 | copies | 25 | copies | 25 | copies | 25 | copies |
| (ATCC, Cat. No. 33152D-5) | | | | | | | | |

Six replicates were performed for each experimental condition.

The reaction cartridges were inserted into a Spartan Cube® thermal cycling device (Spartan Bioscience, Part No. 01014187) and the following thermal cycling program was performed: 1) Initial denaturation: 102.5° C. for 30 seconds followed by 99° C. for 4.5 minutes and 2) Cycling: 50 cycles of 102.5° C. for 5 seconds and 62° C. for 15 seconds. The final reaction volume in each reaction cartridge was 20 μL.

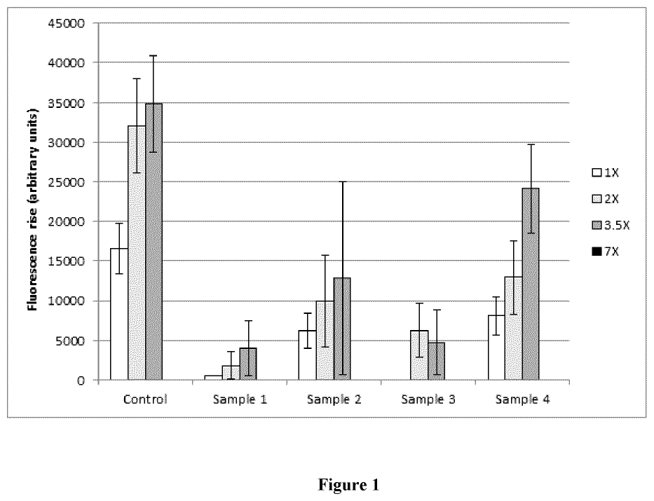

Fluorescence rise (in arbitrary units) for each experimental condition were measured (FIG. 1).

The results show that the 2X and 3.5X conditions resulted in significant fluorescence rises for all four samples indicating that at these conditions, the reagent concentrations were sufficient to overcome any inhibitory factors present in the samples. In contrast, the 1X and 7X conditions failed for some or all samples.

### Example 3: Detection of L. pneumophilia by Spartan qPCR

This example demonstrates the effectiveness of Spartan qPCR for quantifying L. pneumophilia in cooling tower water samples.

The method provided test results in 45 minutes, was performed on-site and thus, did not require shipment of water samples to a central laboratory. In this study, 51 cooling towers were tested for L. pneumophilia weekly using Spartan qPCR and twice per month with laboratory culture. For laboratory culture, cooling tower water samples were shipped to off-site laboratories that performed culture testing according to the ISO 11731 or the CDC culture procedures.

### SUMMARY

Results showed that 8% of cooling towers tested positive for L. pneumophilia with test results greater than 100 GU/mL. 39% of towers tested positive at greater than 10 GU/mL. Overall, 2.2% of results were above 100 GU/mL and 13.3% of were₌greater than 10 GU/mL.

According to the PSPC MD-15161 standard, towers that test positive at greater than 100 GU/mL must be cleaned and disinfected, and their operation and maintenance procedures and chemical treatment program must be reviewed and adjusted. Weekly Spartan qPCR testing identified actionable levels of Legionella 3.5 weeks faster on average than monthly laboratory culture. Of note, 62.5% of results greater than 10 GU/mL, or 10 CFU/mL, were falsely identified as negative by laboratory culture due to bacterial degradation during shipping. In addition, it was observed that Legionella could grow rapidly in cooling towers: 42% of samples grew to a higher action level within 7 days, as categorized by the PSPC MD-15161 standard.

### Methods

Spartan qPCR was performed following concentration of bacteria on a 0.45 um polyethersulfone (PES) filter. The live bacteria were recovered from the filter and eluted into a qPCR cartridge quantification of the DNA by qPCR. Greater than 98% of the free-floating DNA from dead bacteria passed through the filter and was not measured. Results were obtained within 45 minutes. A correction for the number of live bacteria recovered following filtration was applied to the test results so that 1 CFU/mL is equivalent to 1 GU/mL. The limit of detection was 8 GU/mL across a range of cooling tower water samples. Precision of the method was determined by spiking known concentrations of Legionella bacteria into water samples and then performing the method. The pooled standard deviation (SD) from four operators was 0.13 log. This was consistent with the 0.1-0.3 log SD range observed in a study of inter/intra-lab qPCR reproducibility (Baume et al., J. Appl. Microbiol. (2013) 114:1725-1733).

Reproducibility of Spartan qPCR results was demonstrated by testing water samples from 9 cooling towers. Tests were repeated 6 times for each water sample. Results are shown in Table 17.

TABLE 17
Reproducibility of Spartan qPCR

| | | Time Delay | | |
|---|---|---|---|---|
| | On-site | Result | Time Delay | |
| | Result | (GU/mL) | SD | Fold |
| Site ID | (GU/mL) | n = 6 | n = 6 | Change |
| NCA12-1 | <LOD | <LOD | — | — |
| NCA12-3 | <LOD | <LOD | — | — |
| NCA12-4 | <LOD | <LOD | — | — |
| NCA17 | <LOD | <LOD | — | — |
| NCA25-4 | <LOD | <LOD | — | — |
| NCA42 | <LOD | <LOD | — | — |
| O9 | 32 | <LOD | — | −3.2X |
| O10 | 64 | 370 | 49 | 5.8X |
| O11 | 130 | 350 | 76 | 2.7X |

### Study Description

The study described in this example had three main objectives: 1) to determine if there is a correlation between on-site Spartan qPCR and off-site laboratory culture quantification, 2) to determine whether weekly on-site Spartan qPCR leads to a statistically significant improvement in identifying elevated levels of L. pneumophila in comparison to monthly laboratory culture and 3) to validate the accuracy of on-site Spartan qPCR compared to off-site laboratory qPCR Testing.

Test results for qPCR and culture testing were categorized according to action levels presented in Table 3. Categories were derived from a combination of laboratory culture and laboratory qPCR action levels found in PSPC MD-15161.

TABLE 3
Categorization of test results
Level (GU/mL)
<10
10-100
101-1000
>1000

qPCR results are measured according to Genomic Units per milliliter (GU/mL). GU/mL is equivalent to Genomic Equivalents per milliliter (GE/mL). Culture test results were measured according to Colony Forming Units per milliliter (CFU/mL). Legionnaires' disease outbreaks linked to cooling towers typically occur at Legionella levels greater than 100 CFU/mL (Bartram, J., (2007) World Health Organization Geneva).

### Results Summary

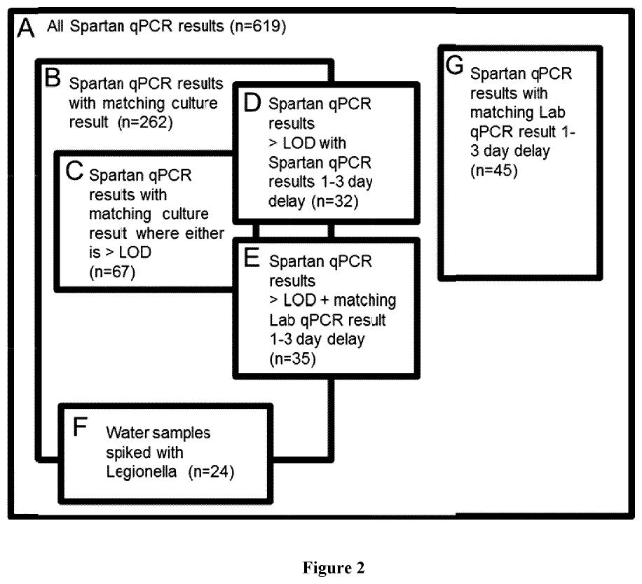

51 cooling towers were tested weekly for 12 weeks using Spartan qPCR. The data collected and analyzed during the study were summarized as shown in FIG. 2 and Table 4.

TABLE 4
Summary of Spartan qPCR results from the 12-week study

| Level (GU/mL) | Test (n) | % Total |
|---|---|---|
| No result | 44 | 7.1 |
| Undetectable | 444 | 7.2 |
| <10 | 52 | 8.4 |
| 10-100 | 65 | 11 |
| 101-1000 | 13 | 2.1 |
| >1000 | 1 | 0.16 |

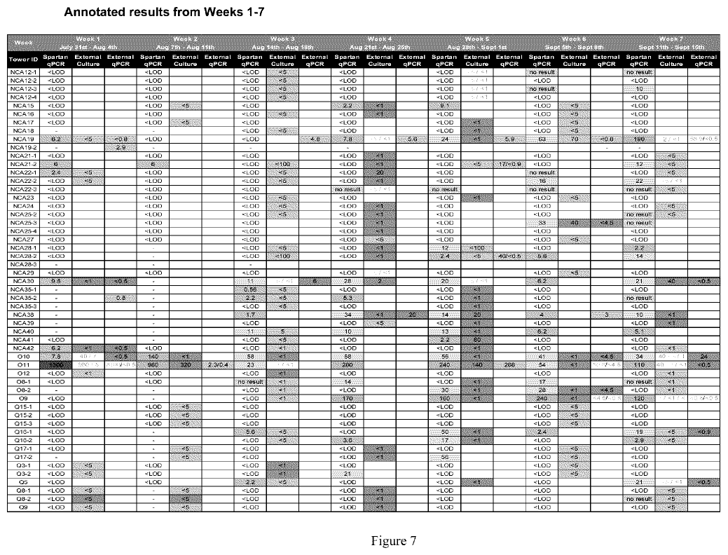

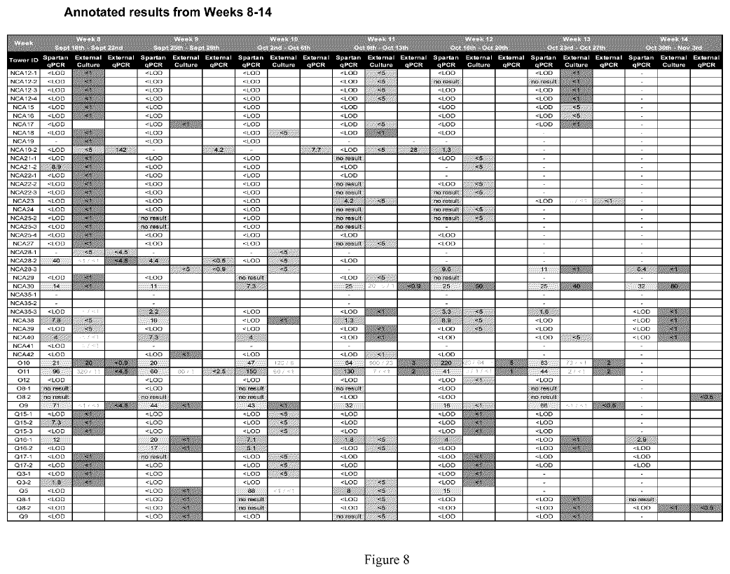

Approximately 13% (79 out of 619) of all Spartan qPCR tests detected L. pneumophila levels greater than 10 GU/mL. 7% (44) of Spartan qPCR tests were unable to produce a result (FIGS. 7 and 8). This was likely due to PCR inhibitors in the water samples and was comparable to other studies using laboratory-based qPCR testing (Diaz-Flores et al. BMC Microbiol. (2015) 15:91; Joly et al., Appl. Environ. Microbiol. (2006) 72:2801-2808).

When Spartan qPCR results were grouped by cooling tower (Box A in FIG. 2), it was observed that 39% of cooling towers (20 out of 51) reached a maximum level greater than 10 GU/mL over the course of the 12-week study (Table 5). 8% of towers (4 out of 51) reached a level greater than 100 GU/mL over 12 weeks. According to the PSPC MD-15161 standard, this is the level at which a cooling tower must be cleaned and disinfected.

TABLE 5
Maximum L. pneumophila level reached
per cooling tower over the 12-week Study

| Level (GU/mL) | Towers (n) | % Total |
|---|---|---|
| <10 | 31 | 61 |
| 10-100 | 16 | 31 |
| 101-1000 | 3 | 6 |
| >1000 | 1 | 2 |

### 1: Correlation of Spartan qPCR and Laboratory Culture Quantification

Spartan qPCR was performed on site and results were available in 45 minutes. In contrast, culture testing took 1-3 days to ship a water sample to a laboratory and 10-14 days to grow the Legionella bacteria. To grow the bacteria, laboratories followed either ISO 11731 “Water quality—Enumeration of Legionella” (International Organization for Standardization, 2017) or the CDC's “Procedures for the Recovery of Legionella from the Environment” (Centers for Disease Control and Prevention (CDC), 2005).

According to the PSPC MD-15161 standard, cooling towers should be tested with laboratory culture every 4 weeks. In this study, frequency of culture testing was increased to approximately every 2 weeks in order to evaluate the potential benefits of early detection.

There were a total of 262 water samples that had both a Spartan qPCR result and a paired laboratory culture result that had been tested in parallel (Table 4 and Box B in FIG. 2). Results below 10 GU/mL or 10 CFU/mL were compared to results above 10 GU/mL or 10 CFU/mL. The concordance rate was 84%: 21 samples had values greater than 10 Gu/mL by both Spartan qPCR and lab culture and 198 samples were below 10 GU/mL by both methods. The discordance rate was 16%: 3 samples (1%) were below 10 GU/mL by Spartan qPCR but above 10 CFU/mL by lab culture and 40 samples (15%) were above 10 GU/mL by Spartan qPCR but below 10 CFU/mL by lab culture. Thus, 62.5% (40/64) of positive results greater than 10 GU/mL or 10 CFU/mL were missed by laboratory culture.

TABLE 6
Concordance of Spartan qPCR and laboratory culture

| | Spartan qPCR | Spartan qPCR |
|---|---|---|
| | (>10 GU/mL) | (<10 GU/mL) |
| Lab culture | 21 | 3 |
| (>10 CFU/mL) | | |
| Lab culture | 40 | 198 |
| (<10 CFU/mL | | |

### Degradation or Growth Due to Shipping

To demonstrate that the shipping time to transport a water sample to a laboratory caused bacterial growth in some samples and bacterial degradation in the other of samples, and that this was the root cause of the 16% discordance rate, three data sets from this study were analyzed. The data sets included samples which were tested with on-site Spartan qPCR and in parallel with laboratory culture, or qPCR, after a 1-3 day shipping delay. The three data sets were:

1. On-site Spartan qPCR vs. delayed laboratory culture

2. On-site Spartan qPCR vs. delayed Spartan qPCR

3. On-site Spartan qPCR vs. delayed laboratory qPCR

LOD was chosen as the cut-off point for the data sets because bacterial levels below 10 GU/mL can still affect the growth or degradation of Legionella bacteria.

### On-Site Spartan qPCR vs. Delayed Laboratory Culture

In order to assess the correlation between Spartan qPCR and laboratory culture, 67 results that were greater than LOD by on-site Spartan qPCR or laboratory culture with a 1-3 day shipping delay were compared (Box C in FIG. 2). The effect of delay was classified as unchanged (less than 2-fold change or less than LOD between the two test results), bacterial growth (greater than 2-fold increase), and bacterial degradation (greater than 2-fold decrease). The results were: unchanged (22%; 15/67), bacterial growth (12%; 8/67), and bacterial degradation (67%; 45/67) (Table 7). Note that unchanged is annotated as

TABLE 7
On-site Spartan qPCR vs. delayed laboratory culture

| Spartan | Delayed Lab | | |
|---|---|---|---|
| qPCR | Culture | Time Delay | |
| (GU/mL) | (CFU/mL) | (Days) | Effect of Delay |
| 1300 | 960 | 3 | — |
| 980 | 320 | 1 | Degradation |
| 240 | <1 | 2 | Degradation |
| 220 | 520 | 1 | Growth |
| 190 | <1 | 1 | Degradation |
| 160 | <1 | 2 | Degradation |
| 150 | 60 | 1 | Degradation |
| 140 | <1 | 1 | Degradation |
| 130 | 7 | 2 | Degradation |
| 120 | <1 | 0 | Degradation |
| 110 | 40 | 2 | Degradation |
| 96 | 320 | 2 | Growth |
| 88 | <1 | 2 | Degradation |
| 83 | 73 | 1 | — |
| 71 | <1 | 2 | Degradation |
| 66 | <1 | 2 | Degradation |
| 64 | 500 | 2 | Growth |
| 63 | 70 | 1 | — |
| 60 | 80 | 2 | — |
| 58 | <1 | 3 | Degradation |
| 56 | <1 | 3 | Degradation |
| 54 | <1 | 1 | Degradation |
| 50 | <1 | 3 | Degradation |
| 47 | 120 | 1 | Growth |
| 44 | <1 | 2 | Degradation |
| 44 | 2 | 1 | Degradation |
| 43 | <1 | 2 | Degradation |
| 41 | <1 | 1 | Degradation |
| 41 | 9 | 1 | Degradation |
| 40 | <1 | 2 | Degradation |
| 34 | 40 | 2 | — |
| 34 | <1 | 1 | Degradation |
| 33 | 40 | 2 | — |
| 32 | 80 | 2 | Growth |
| 30 | <1 | 2 | Degradation |
| 28 | 2 | 1 | Degradation |
| 28 | <1 | 2 | Degradation |
| 25 | 40 | 2 | — |
| 25 | 20 | 1 | — |
| 25 | 50 | 2 | — |
| 24 | <1 | 2 | Degradation |
| 23 | <1 | 0 | Degradation |
| 22 | <5 | 1 | Degradation |
| 21 | 40 | 2 | — |
| 21 | 20 | 2 | — |
| 21 | <5 | 2 | Degradation |
| 20 | <5 | 0 | Degradation |
| 20 | <1 | 2 | Degradation |
| 19 | <5 | 0 | Degradation |
| 17 | <1 | 3 | Degradation |
| 17 | <1 | 2 | Degradation |
| 16 | <1 | 0 | Degradation |
| 14 | 20 | 2 | — |
| 14 | <1 | 2 | Degradation |
| 13 | <1 | 2 | Degradation |
| 12 | <5 | 1 | Degradation |
| 11 | <5 | 1 | Degradation |
| 11 | 5 | 1 | Degradation |
| 11 | <1 | 2 | Degradation |
| 10 | <1 | 2 | Degradation |
| 9.6 | <1 | 1 | Degradation |
| 8.9 | <5 | 1 | — |
| 8.9 | <1 | 1 | Degradation |
| 8 | <5 | 1 | — |
| 7.8 | 40† | 1 | Growth |
| 2.2 | 80* | 2 | Growth |
| <LOD | 20‡ | 1 | Growth |

Three samples that were less than 10 GU/mL for on-site Spartan qPCR and greater than 10 CFU/mL for laboratory culture were analyzed (these samples are the last three entries in Table 7). Each sample came from a different cooling tower. For one tower (*), the culture result of 80 CFU/mL was the only culture-positive result over the course of the 12-week study. For the second tower (†), Spartan qPCR test results were also positive in subsequent weeks. This indicated that water from that cooling tower was conducive to bacterial growth. For both of these samples, Spartan qPCR detected L. pneumophila, but at levels much lower than by laboratory culture. The third tower (‡) had been positive 3 weeks earlier for L. pneumophila, but at a concentration less than 10 GU/mL. In all three instances, low levels of bacteria in the water sample experienced growth during shipping to the laboratory.

### On-Site Spartan qPCR vs. Delayed Spartan qPCR

As a second test of the effect of shipping, 32 water samples that were greater than LOD by on-site Spartan qPCR were tested again by Spartan qPCR after a 1-3 day time delay. These samples are labelled as Box D in FIG. 2. Results are shown in Table 8.

TABLE 8
On-site qPCR v. delayed Spartan qPCR

| | Delayed | | |
|---|---|---|---|
| Spartan | Spartan | | |
| qPCR | qPCR | Time Delay | |
| (GU/mL) | (GU/mL) | (days) | Effect of Delay |
| 220 | 270 | 1 | — |
| 150 | <LOD* | 2 | Degradation |
| 130 | <LOD* | 2 | Degradation |
| 96 | <LOD* | 2 | Degradation |
| 88 | 0.67 | 3 | Degradation |
| 83 | 67 | 1 | — |
| 71 | <LOD | 3 | Degradation |
| 66 | 14 | 2 | Degradation |
| 64 | <LOD* | 2 | Degradation |
| 60 | 13 | 2 | Degradation |
| 47 | <LOD* | 2 | Degradation |
| 44 | <LOD | 3 | Degradation |
| 44 | 1.6 | 1 | Degradation |
| 43 | <LOD | 3 | Degradation |
| 40 | <LOD | 1 | Degradation |
| 32 | <LOD | 1 | Degradation |
| 25 | <LOD | 1 | Degradation |
| 25 | 8 | 2 | Degradation |
| 25 | 26 | 1 | — |
| 21 | <LOD | 3 | Degradation |
| 20 | 15 | 2 | — |
| 17 | <LOD* | 2 | Degradation |
| 16 | <LOD* | 1 | Degradation |
| 16 | <LOD | 2 | Degradation |
| 15 | <LOD | 3 | Degradation |
| 14 | 25 | 1 | — |
| 12 | <LOD | 2 | Degradation |
| 11 | <LOD | 1 | Degradation |
| 11 | 26 | 1 | Growth |
| 7.3 | 32 | 1 | Growth |
| 7.3 | 19 | 1 | Growth |
| 4 | 39 | 1 | Growth |

*indicates L. pneumophila was detected but did not pass quantification metrics and therefore was deemed to be less than the Limit of Detection (<LOD).

16% ( 5/32) of samples were unchanged (less than 2-fold change or less than LOD). In contrast, 13% ( 4/32) of samples showed bacterial growth (greater than 2-fold increase) and 72% ( 23/32) showed bacterial degradation (greater than 2-fold decrease). Of note, 40% of samples started off at a value greater than 10 GU/mL and decreased to less than LOD following the time delay. qPCR was an extremely sensitive DNA detection technique and it was remarkable that the DNA was completely degraded and undetectable in these samples after only 1-3 days.

Results from these data show that a time delay can lead to bacterial growth or degradation, depending on the water sample. Similar to the first data set, this indicated that discordance between on-site Spartan qPCR and laboratory culture was primarily due to the effect of shipping delay.

### On-Site Spartan qPCR vs. Delayed Laboratory qPCR

35 water samples that were greater than LOD by on-site Spartan qPCR or laboratory qPCR following a 1-3 day shipping delay were analyzed. These 35 samples are labelled as Box E in FIG. 2. Results are shown in Table 9.

TABLE 9
On-site Spartan qPCR vs. delayed laboratory qPCR

| Spartan | Delayed Lab | | |
|---|---|---|---|
| qPCR | qPCR | Time Delay | |
| (GU/mL) | (GU/mL) | (days) | Effect of Delay |
| 1300 | <0.5 | 3 | Degradation |
| 980 | 2.3 | 1 | Degradation |
| 240 | 288 | 0 | — |
| 240 | <4.5 | 3 | Degradation |
| 220 | 5 | 2 | Degradation |
| 190 | 58.9 | 1 | Degradation |
| 130 | 2 | 3 | Degradation |
| 120 | <0.8 | 0 | Degradation |
| 110 | <0.5 | 2 | Degradation |
| 96 | <4.5 | 2 | Degradation |
| 83 | 2 | 1 | Degradation |
| 71 | <4.5 | 2 | Degradation |
| 66 | <0.5 | 5 | Degradation |
| 64 | 3 | 3 | Degradation |
| 63 | <0.8 | 1 | Degradation |
| 60 | <2.5 | 7 | Degradation |
| 54 | 50.6 | 0 | — |
| 44 | 2 | 1 | Degradation |
| 41 | <4.5 | 2 | Degradation |
| 41 | 1 | 2 | Degradation |
| 40 | <4.5 | 3 | Degradation |
| 34 | 24 | 2 | — |
| 34 | 20 | 1 | — |
| 33 | 4.5 | 1 | Degradation |
| 28 | <4.5 | 2 | Degradation |
| 25 | <0.9 | 3 | Degradation |
| 24 | 5.9 | 1 | Degradation |
| 21 | <0.5 | 3 | Degradation |
| 21 | <0.5 | 2 | Degradation |
| 21 | <0.9 | 2 | Degradation |
| 19 | <0.9 | 1 | Degradation |
| 11 | 6 | 1 | — |
| 2.4 | 40 | 1 | Growth |
| <LOD | 17 | 1 | Growth |
| <LOD | 28 | 1 | Growth |

As show in the prior two data sets, 9% (3/35) of samples were unchanged (less than 2-fold change or less than LOD). In contrast, 14% (5/35) of samples showed bacterial growth (greater than 2-fold increase) and 77% (27/35) showed bacterial degradation (greater than 2-fold decrease). This data set also indicated that the discordance between on-site Spartan qPCR and laboratory culture was primarily due to the effect of shipping delay.

### Time Delay Effects in Three Data Sets

Overall, three data sets were analyzed to compare on-site Spartan qPCR versus testing with a time delay of 1-3 days. All three data sets demonstrated a significant effect of time delay on quantification, with bacterial degradation being the most common effect (Table 10).

TABLE 10
Time delay effects in three data sets

| Relative | Delayed | Delayed | Delayed |
|---|---|---|---|
| Change | Laboratory | Spartan | Laboratory |
| Over Time | Culture (%) | qPCR (%) | qPCR (%) |
| Degradation | 67 | 72 | 77 |
| Unchanged | 22 | 16 | 9 |
| Growth | 12 | 13 | 14 |

### Direct qPCR vs. Delayed Direct qPCR

To further test the effects of shipping on test results, cooling tower water samples were spiked with known amounts of live L. pneumophila and tested in a laboratory before and after time delays of 24, 48, and 72 hours. The water samples included seven that had tested positive in the field and 17 that had tested negative (Box F in FIG. 12). The spiked samples were treated with different simulated shipping conditions: storage temperatures of 20° C. or 37° C. and storage conditions with and without sodium thiosulfate (Table 11). Sodium thiosulfate was added to water samples to neutralizes chlorine and minimizes bacterial degradation during shipping.

TABLE 11
Simulated shipping conditions (temperature and sodium thiosulfate)

| Storage | | |
|---|---|---|
| Temperature | Sodium | Samples |
| (° C.) | thiosulfate | (n) |
| 20 | Yes | 24 |
| 37 | Yes | 23* |
| 20 | No | 22* |

*3 samples were lost due to circumstances unrelated to testing.

At the laboratory, samples were tested with direct qPCR for L. pneumophila. Direct qPCR removed the potential confounding effect of bacterial loss due to filtration and measured levels of DNA directly. Results of this experiment are shown in Table 12. The values at 24, 48, and 72 hours were expressed as a percent of the DNA concentration at time 0 hours. There were no significant differences between samples stored at 20° C. or 37° C., or treated with or without sodium thiosulfate.

TABLE 12
Change in L. pneumophila levels over 72 hours

| Relative Change | | | |
|---|---|---|---|
| Over Time | 24 h (%) | 48 h (%) | 72 h (%) |
| Bacterial degradation | 38 | 55 | 65 |
| No change | 58 | 36 | 30 |
| Bacterial growth | 4 | 9 | 4 |

Similar to the previous three data sets, 30% of samples were unchanged at 72 hours (less than 2-fold change or less than LOD). In contrast, 4% of samples showed bacterial growth (greater than 2-fold increase) and 65% showed bacterial degradation (greater than 2-fold decrease).

These results indicate that a shipping delay can lead to bacterial growth or degradation, depending on the water sample. These results also demonstrate that Legionella DNA can degrade in as few as 24 hours. Of note, sodium thiosulfate did not significantly decrease bacterial degradation. Based on the consistency of results across the four data sets, shipping time and conditions explain the discordant results between on-site Spartan qPCR and laboratory culture.

### Discussion

This study demonstrated that on-site Spartan qPCR was more sensitive than laboratory culture. Specifically, 62.5% of results greater than 10 GU/mL or 10 CFU/mL were falsely identified as negative by laboratory culture due to bacterial degradation during shipping (Table 6). Instead of attributing this discordance to qPCR detecting dead bacteria, two alternative mechanisms: i) bacterial degradation of water samples during shipping, and ii) culture pre-treatments such as filtration, acid, and heat decreased the viability of Legionella and leading to lower colony counts have been demonstrated.

The time delay for shipping water samples to laboratories lead to bacterial growth in a minority of samples and bacterial degradation in a majority of samples. With Spartan qPCR, 66-77% of samples experienced degradation due to e.g., presence of biocides in the shipped water samples. Biocides are known to inhibit qPCR tests and higher levels of biocides would be expected to lead to higher levels of inhibition and more “no result” tests.

In some instances qPCR may be more sensitive than bacterial culture because qPCR is detecting the DNA of dead, non-pathogenic bacteria that do not grow in culture. However, Spartan qPCR included a step to filter out free DNA and capture of living cells. This was demonstrated by the finding that direct qPCR (no filtering step) resulted in quantification values approximately 2-fold higher than Spartan qPCR (Table 18). The concordance rate between Spartan qPCR and laboratory culture was 84% (Table 6) and the discordant results were fully explained by bacterial growth or bacterial degradation due to shipping time to the laboratory (Table 10). Thus, Spartan qPCR and laboratory culture detected live bacteria when not confounded by bacterial degradation due to shipping time.

### 2: Weekly Spartan qPCR vs. Monthly Laboratory Culture

### Improved Time to Action

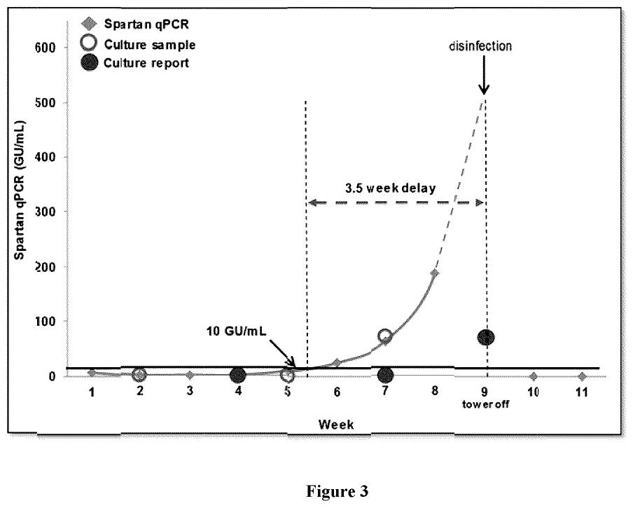

This example also demonstrated that weekly on-site Spartan qPCR resulted in a statistically significant improvement in identifying elevated levels of L. pneumophilia in comparison to monthly laboratory culture. This analysis was based on the test results in Box A, FIG. 2. The method of calculating the improvement is depicted in FIG. 3. In brief, time 0 was calculated as the point where the interpolated Spartan qPCR values equaled 10 GU/mL. From this point, the time to action was calculated for weekly Spartan qPCR and monthly laboratory culture testing.

There were 14 instances in which Spartan qPCR led to faster time to action. Overall, the results showed that weekly Spartan qPCR was 3.5 weeks faster on average than monthly laboratory culture for identifying when L. pneumophila levels exceeded 10 GU/mL (Table 13). The difference of 3.5 weeks was highly statistically significant (p<0.001) as calculated by a two-sided Student's t-test with unequal variances.

TABLE 13
On-site Spartan qPCR for early detection of L. pneumophila

| | Spartan | Culture |
|---|---|---|
| Tower | Delay | Delay |
| NCA19 | 0.6 | 2.8 |
| NCA25-3 | 1.6 | 3.8 |
| NCA28-2 | 0.4 | 6* |
| NCA30 | 0.6 | 5.4 |
| NCA30 | 0.8 | 3 |
| NCA38 | 0.4 | 3.2 |
| O10 | 1.8 | 3.2 |
| O10 | 0.9 | 2.9 |
| O10 | 0.9 | 2.7 |
| O10 | 0.9 | 2.7 |
| O9 | 0.6 | 6* |
| O11 | 1 | 6* |
| Q16.1 | 0.6 | 6* |
| Q5 | 0.2 | 6* |
| Average | 0.8 | 4.3 |

4.3 − 0.8 = 3.5 weeks faster time to action with Spartan
*If culture results were not positive during the time period, the culture delay was set at 6 weeks. This corresponded to te regularly scheduled culture frequency of 4 weeks plus the 2-week delay to grow the bacteria and get a result.

### Weekly Testing

Weekly performance of Spartan qPCR provided an early detection advantage of 3.5 weeks vs. monthly laboratory culture. Thirt-three instances in which Spartan qPCR results increased by 2-fold or more from one week to the second week (and where the second result was greater than 10 GU/mL) were tabulated to determine whether weekly testing was an appropriate frequency (Box A in FIG. 2).

Nine cooling towers had growth between 11-fold and 170-fold over 7 days. The effect of testing every week, or testing every 2 weeks, was also analyzed (Tables 14 and 15). With weekly testing, 42% ( 33/79) of positive events increased to a higher action level within 7 days. With testing every 2 weeks, 52% ( 41/79) of positive events increased to a higher action level within 14 days.

TABLE 14
Changes in action levels with testing every week

| Week n-1 | Week n | Events | Percent of |
|---|---|---|---|
| (GU/mL) | (GU/mL) | (n) | positives(%) |
| <10 | 10-100 | 25 | 32 |
| <10 | 101-1,000 | 2 | 3 |
| <10 | >1,000 | 1 | 1 |
| 10-100 | 101-1,000 | 5 | 6 |
| 10-100 | >1,000 | 0 | 0 |
| 101-1,000 | >1,000 | 0 | 0 |
| | Total | 33 | 42% |

TABLE 15
Changes in action levels with testing every 2 weeks

| | | | Percent of |
|---|---|---|---|
| Week n-2 | Week n | | positives |
| (GU/mL) | (GU/mL) | Events (n) | (%) |
| <10 | 10-100 | 31 | 39 |
| <10 | 101-1,000 | 4 | 5 |
| <10 | >1,000 | 1 | 1 |
| 10-100 | 101-1,000 | 5 | 6 |
| 10-100 | >1,000 | 0 | 0 |
| 101-1,000 | >1,000 | 0 | 0 |
| | Total | 41 | 52% |

According to the PSPC MD-15161 standard, no action is required for test results <10 GU/mL. For test results greater than 10 but less than 100 GU/mL, a cooling tower's Operation & Maintenance (O&M) and Water Treatment Program should be reviewed and adjusted. For test results greater than 100 GU/mL, a cooling tower must be cleaned and disinfected, and the O&M and Water Treatment Program should be reviewed and adjusted. As demonstrated here, if testing is performed every 2 weeks instead of weekly, 42% of positive samples would not be acted upon for an additional week.

### Discussion

A rapid growth rate of L. pneumophila was seen in this study and was consistent with other studies. Under optimal growth conditions, the doubling time of L. pneumophila was found to be 99 minutes (Ristroph et al., J. Clin. Microbiol. (1980) 11:19-21). In water systems and the natural environment, the doubling time is typically between 22-72 hours (French Ministry of the Environment, ARIA No. 19456 (2006)). However, the doubling time at an “amplifier site” (such as a cooling tower) can be as few as 150 minutes, as reported in a case to investigators from the American Society of Heating, Refrigerating and Air-Conditioning Engineers (ASHRAE) (Marshall and Bellucci, Hosp. Rev. (1986) 4).

Since 42% of positive L. pneumophila samples grew to a higher action level within 7 days (Table 14), this study demonstrated that weekly testing is appropriate.

### 3: On-Site Spartan qPCR vs. Laboratory qPCR

The third objective of the study was to determine how on-site Spartan qPCR compared to laboratory qPCR testing. Similar to laboratory culture, laboratory qPCR required 1-3 days for shipment of a water sample to an off-site laboratory. In contrast, on-site Spartan qPCR was performed on a water sample with no shipping delay.

### Spartan qPCR v. Laboratory qPCR using Spiked Water Samples after a 24 Hour Delay

To compare Spartan qPCR v. laboratory qPCR, the performance of both tests was evaluated following a 24 hour shipping delay using sterile water samples spiked with 27 or 80 Gu/mL of live L. pneumophilia (3 replicates per condition). The original concentrations of the spiked bacteria were determined using direct qPCR to avoid introducing the variable of DNA loss from filtration. Sterile water was used to avoid introducing the variable of cooling tower chemicals or substances that could lead to bacterial degradation or growth during shipping.

Water samples were shipped according to recommended conditions for the qPCR laboratory, and all samples arrived with 24 hours.

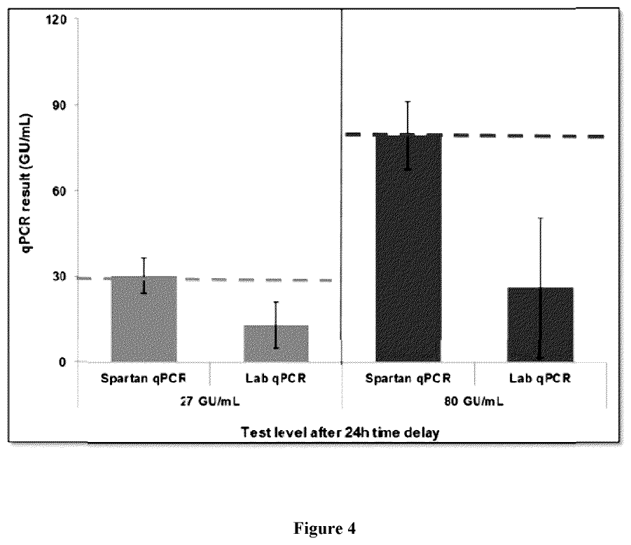

Results showed that Spartan qPCR accurately quantified the bacteria, whereas laboratory qPCR generated results that were approximately 2-fold lower than the known input concentrations (FIG. 4). This demonstrated that Spartan qPCR results are more accurate than laboratory qPCR, due to, e.g., correction for bacteria lost during filtration.

### Spartan qPCR vs. Laboratory qPCR for Real-Life Water Samples

A further comparison between Spartan qPCR and laboratory qPCR was performed by tabulating results from 45 cooling tower water samples that were analyzed by both methods (Box G in FIG. 2). Spartan qPCR was performed on-site and laboratory qPCR was performed following a 1-3 day shipping delay. Results demonstrated a concordance rate of 36% between Spartan qPCR and laboratory qPCR (Table 14). This was significantly lower than the 84% concordance rate between Spartan qPCR and laboratory culture and appeared to be due to shipping delay.

TABLE 16
Spartan qPCR vs. laboratory qPCR for real-life water samples

| | Spartan qPCR | Spartan qPCR |
|---|---|---|
| | (>10 GU/mL) | (<10 GU/mL) |
| Lab qPCR | 6 | 3 |
| (>10 CFU/mL) | | |
| Lab qPCR | 26 | 10 |
| (<10 CFU/mL | | |

### Laboratory qPCR vs. Laboratory Culture for Real-Life Water Samples

To further investigate the low concordance between Spartan qPCR and laboratory qPCR, laboratory qPCR vs. laboratory culture for 43 cooling tower water samples that were shipped from the study sites were compared. The culture laboratories followed ISO 11731 or the CDC's procedures. The qPCR laboratories followed ISO 12869:2012. Table 17 shows the results.

TABLE 17
Laboratory qPCR vs. laboratory culture for real-life water samples

| | Lab qPCR | Lab qPCR |
|---|---|---|
| | (>10 GU/mL) | (<10 GU/mL) |
| Lab culture | 3 | 12 |
| (>10 CFU/mL) | | |
| Lab culture | 7 | 21 |
| (<10 CFU/mL | | |

Concordance rate between laboratory qPCR and laboratory culture was 56%. This was lower than the 84% concordance rate between Spartan qPCR and laboratory culture (Table 6). This indicated that laboratory qPCR fails to detect a significant number of positive samples. Overall, the results indicated that on-site Spartan qPCR provides better concordance with laboratory culture than laboratory qPCR. Laboratory qPCR results are affected by bacterial degradation during shipping and loss of bacteria due to filtration, and these factors and lead to under-calling of L. pneumophila.

The concordance rate was 84% between Spartan qPCR and laboratory culture, and only 56% between laboratory qPCR and laboratory culture (Tables 6 and 17). Results indicated that laboratory qPCR failed to detect a significant number of positive samples. The reasons included (a) shipping delay and bacterial degradation, (b) lower bacterial recovery rates for laboratory qPCR, (c) negative impact from biocides in the water samples. In contrast, Spartan qPCR was performed with no shipping delay and was designed to correct for bacterial recovery rates.

### Discussion

In this study, 51 cooling towers were tested weekly, over a 12-week period, with Spartan qPCR and 13.3% of tests had levels of L. pneumophila greater than 10 GU/mL (Table 4). The towers were also tested weekly using dipslides and twice per month using laboratory culture. Over the course of the study, 8% of cooling towers had L. pneumophila levels greater than 100 GU/mL and 39% of towers had levels greater than 10 GU/mL (Table 5). For the 8% (4 out of 51) of cooling towers, 3 of the 4 failed to identify the elevated L. pneumophila levels with monthly laboratory culture. These findings demonstrate that cooling towers following the current PSPC MD-15161 standard for biocide treatment and Legionella monitoring continue to be at risk of Legionella growth.

Spartan qPCR is performed on-site. In contrast, laboratory culture and laboratory qPCR are performed after a shipping delay for the water samples. This study showed that both laboratory culture and laboratory qPCR results were affected by L. pneumophila growth or degradation during shipping (Table 10). Specifically, 15% of Spartan qPCR results were falsely identified as negative by culture due to bacterial degradation during shipping (Table 6).

When performed weekly, Spartan qPCR provided an early detection advantage of 3.5 weeks on average vs. monthly laboratory culture for L. pneumophila levels greater than 10 GU/mL (Table 13). Weekly testing was shown to be important because 42% of positive L. pneumophila samples grew to a higher action level within 7 days (Table 14).

### Example 4: Cooling Tower with >1000 GU/mL of L. pneumophilia

This example demonstrated identification of a cooling tower that tested greater than 1,000 GU/mL by Spartan qPCR. This result was confirmed with different laboratory methods. Regularly-scheduled laboratory culture and dipslide testing failed to identify actionable levels of Legionella in this tower at the time of Spartan qPCR testing and in subsequent weekly testing.

### Initial Testing

Cooling tower O11 tested positive for L. pneumophila at 1,300 GU/mL by Spartan qPCR (Table 18). Direct qPCR testing of the water sample at a laboratory using a mainframe DNA analyzer after 2 days and 3 days of storage resulted in values of 3,100 GU/mL and 3,300 GU/mL, respectively. In parallel, the water sample that had been stored for 2 days was sent to a second qPCR laboratory for testing. The second laboratory reported a result of less than 0.5 GU/mL. A dipslide test result was negative (less than 10,000 Total Bacterial Count). A third qPCR laboratory tested the sample and reported a result of 8,100 GU/mL.

TABLE 16
Test results for cooling tower O11

| Test Type | Week 1 | Week 2 | Week 3 | Week 4 | Week 5 |
|---|---|---|---|---|---|
| Spartan qPCR (GU/mL) | 1,300† | 980 | 23 | 280 | 240 |
| Direct qPCR (GU/mL) | 3,100†/73,300* | 11‡/160* | — | 730* | — |
| Spartan Direct Culture (CFU/mL) | 11,000* | 2,000‡ | <4‡ | — | — |
| Lab qPCR (GU/mL) | <0.5‡/8,100 | 6*/2.3 | — | — | 288 |
| Lab #1 Culture (CFU/mL) | 5‡ | — | <1 | — | — |
| Lab #2 Culture (CFU/mL) | 960* | 320* | <1* | Lost in | 140‡ |
| | | | | shipping | |

‡time delay of 1 day;
‡time delay of 2 days;
*time delay of 3 or more days.

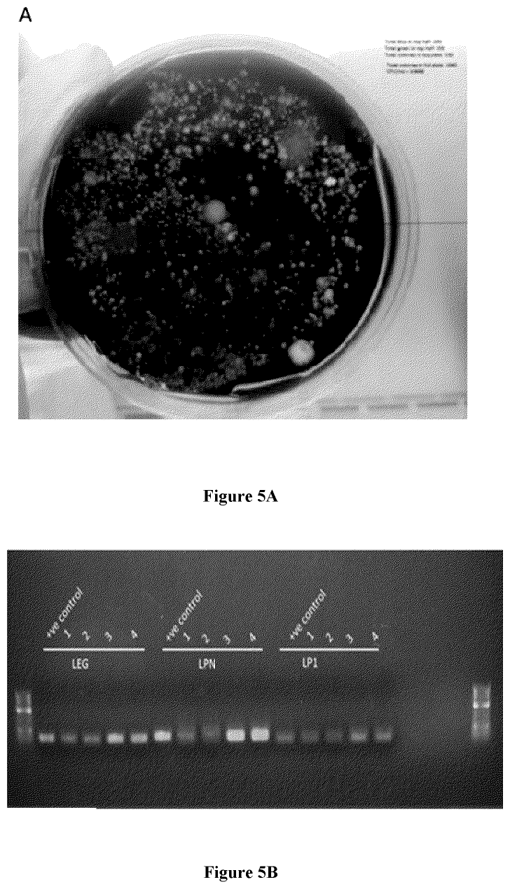

During Week 1, the cooling tower's regularly-scheduled laboratory culture gave a value of 5 CFU/ml. In parallel, Spartan direct culture testing on the water sample determined a value of 11,000 CFU/mL (FIG. 5A). Spartan's direct culture was performed by direct plating i.e., the water sample was plated directly without filtering or concentration. A third laboratory cultured a water sample and determined a value of 960 CFU/mL. Culture values between the two third-party culture laboratories were widely different, possible due to differing culture methods that were used.

Biocide levels in the cooling tower were adjusted and Spartan qPCR results decreased to 23 GU/mL by week 3. By week 4 bacterial levels increased to 280 GU/mL by Spartan qPCR and remained elevated over the course of the 12 week study.

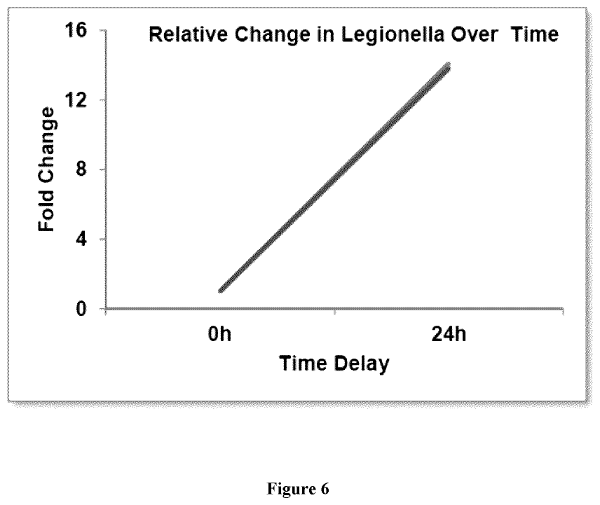

To demonstrate the water sample's capacity to support growth, a water sample from cooling tower O11 was collected at week 7 of the study and spiked with live L. pneumophilia bacteria. Direct qPCR was used to monitor the concentration of L. pneumophila right after begin spiked and 24 hours later, with and without sodium thiosulfate. Results showed that the bacteria grew approximately 14-fold in 24 hours, from 6700 GU/ml to 92600 GU/mL (with sodium thiosulfate) and from 5500 GU/mL to 77200 GU/mL (without sodium thiosulfate) (FIG. 6).

On-site Spartan qPCR at week 8 determined a value of 96 GU/mL, whereas laboratory culture determined value of 320 CFU/mL (following a 2-day shipping delay).

The Spartan qPCR result of 1300 GU/mL was the most accurate as compared to the other methods. Direct qPCR results greater than 3000 GU/mL were likely due to a combination of continued bacterial growth and failure to filter out free DNA. Laboratory qPCR results of <0.5 GU/mL were likely due to bacterial degradation from shipping delay. The laboratory qPCR result of 8100 GU/mL was performed on the same day and therefore not affected by shipping delay. The difference in laboratory culture values (5CFR/mL v. 960 CFU/mL) were likely due to methodological differences between the two laboratories. The direct culture value of 11000 CFU/mL was likely due to bacterial growth during 3 days of storage.

The importance of weekly qPCR testing was demonstrated by this study of a cooling tower where L. pneumophila levels greater than 1,000 GU/mL were missed by weekly dipslides and monthly culture testing (Table 16).

### Equivalents

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

TABLE OF SEQUENCES
(SEQ ID NO: 1)
TTGTCTTATAGCATTGGTGCCG-3′
(SEQ ID NO: 2)
CCAATTGAGCGCCACTCATAG-3′
(SEQ ID NO: 3)
5′-CAL_610-CAATTGAGCGCCACTCATAG-BHQ-2-3′

We claim:

1. A method comprising steps of:
obtaining an environmental sample comprising a microorganism, wherein the microorganism comprises a nucleic acid;
concentrating the environmental sample to produce a concentrated sample, wherein the microorganism is concentrated about 2-fold to about 125-fold in the concentrated sample as compared to the environmental sample;
contacting the concentrated sample with a nucleic acid amplification reagent in a reaction vessel, wherein the concentrated sample is directly contacted with the nucleic acid amplification reagent without any intervening steps; and
performing a nucleic acid amplification reaction on the nucleic acid from the microorganism in the concentrated sample.

2. The method of claim 1, wherein the environmental sample is a water sample selected from the group consisting of industrial cooling tower water, untreated fresh water, waste water, stagnant water, wash water, grey water and water obtained from a lavatory, shower, bathtub, toilet, sink.

3. The method of any one of claims 1 and 2, wherein the microorganism is a bacteria, cyanobacteria, virus, protozoa, fungus or rotifer.

4. The method of claim 3, wherein the bacteria is selected from the group consisting of Alicyclobacillus, Aeromonas, Bacteroides, Bifidobacterium, Campylobacter, Citrobacter, Clostridia, Enterobacter, Enteroccocus, Escherichia, Eubacterium, Klebsiella, Lactobacillus, Legionella, Listeria, Mycobacterium, Pseudomonas, Raoultella, Salmonella, Shigella, Streptococcus, Vibrio and combinations thereof.

5. The method of claim 3 or 4, wherein the bacteria is selected from the group consisting of Legionella pneumophila, Legionella longbeachae, Legionella bozemannii, Legionella micdadei, Legionella feeleii, Legionella dumoffii, Legionella wasdworthii, Legionella anisa and combinations thereof.

6. The method of claim 3 or 4, wherein the bacteria is Escherichia coli.

7. The method of any one of claims 1-6, wherein the environmental sample is concentrated to produce the concentrated sample by filtration, evaporation and/or centrifugation.

8. The method of any one of claims 1-7, wherein the environmental sample is concentrated to produce the concentrated sample by filtration.

9. The method of claim 8, wherein the filtration step comprises washing a retentate and/or eluting the concentrated sample from the filter.

10. The method of claim 8 or 9, wherein the filtration is performed using a hydrophilic filter membrane.

11. The method of any one of claims 8-10, wherein the filtration is performed using a hydrophilic polyethersulfone (PES) filter membrane.

12. The method of any one of claims 1-11, wherein the nucleic acid amplification reaction comprises a DNA polymerase at a concentration of at least 1.0 U/reaction and a primer at a concentration of at least 0.2 μM.

13. The method of any one of claims 1-12, wherein the nucleic acid amplification reaction comprises a probe at a concentration ranging from at least 1.0 μM to about 14 μM.

14. The method of claim 12, wherein the DNA polymerase is at a concentration ranging from at least 3.4 U/reaction to about 45 U/reaction.

15. The method of claim 12, wherein the primer is at a concentration ranging from at least 1.3 μM to about 15 μM.

16. The method of any one of claims 1-15, wherein the nucleic acid amplification reagent does not comprise a reagent which is designed to resist DNA polymerase inhibitors.

17. The method of any one of claims 1-16, wherein the method does not include a step of lysing the microorganism.

18. The method of any one of claims 1-17, wherein the method does not further include a step of purifying the nucleic acid from the microorganism.

19. The method of claim 12, wherein the nucleic acid amplification reaction comprises a DNA polymerase at a concentration ranging from at least 12 U/reaction to about 21 U/reaction, a primer at a concentration ranging from at least 4.0 μM to about 7.0 μM and a probe at a concentration ranging from at least 3.5 μM to about 7.0 μM.

20. The method of any one of claims 1-19, further comprising a step of determining whether an amplification product was produced as a result of the nucleic acid amplification reaction.

21. A method comprising steps of:
obtaining a sample comprising a nucleic acid;
contacting the sample with a nucleic acid amplification reagent in a reaction vessel,
wherein the sample is directly contacted with the nucleic acid amplification reagent without any intervening steps and
wherein the nucleic acid amplification reagent comprises a DNA polymerase at a concentration ranging from at least 6 U/reaction to about 42 U/reaction, a primer at a concentration ranging from at least 2.0 μM to about 14 μM and a probe at a concentration ranging from at least 1.9 μM to about 14 μM; and
performing a nucleic acid amplification reaction on the nucleic acid from the sample.

22. The method of claim 21, wherein the sample is selected from the group consisting of an environmental sample and a biological sample.

23. The method of claim 22, wherein the environmental sample is a concentrated sample.

24. The method of claim 22, wherein the environmental sample is a water sample selected from the group consisting of industrial cooling tower water, untreated fresh water, waste water, stagnant water, wash water, grey water and water obtained from a lavatory, shower, bathtub, toilet, sink.

25. The method of claim 24, wherein the environmental sample comprises a microorganism and wherein the microorganism comprises a nucleic acid.

26. The method of claim 25, wherein the microorganism is a bacteria, cyanobacteria, virus, protozoa, fungus or rotifer.

27. The method of claim 26, wherein the bacteria is selected from the group consisting of Alicyclobacillus, Aeromonas, Bacteroides, Bifidobacterium, Campylobacter, Citrobacter, Clostridia, Enterobacter, Enteroccocus, Escherichia, Eubacterium, Klebsiella, Lactobacillus, Legionella, Listeria, Mycobacterium, Pseudomonas, Raoultella, Salmonella, Shigella, Streptococcus, Vibrio and combinations thereof.

28. The method of claim 26 or 27, wherein the bacteria is selected from the group consisting of Legionella pneumophila, Legionella longbeachae, Legionella bozemannii, Legionella micdadei, Legionella feeleii, Legionella dumoffii, Legionella wasdworthii, Legionella anisa and combinations thereof.

29. The method of claim 27, wherein the bacteria is Escherichia coli.

30. The method of claim 22, wherein the biological sample is selected from the group consisting of a cell sample, a body fluid sample and a swab sample.

31. The method of claim 22, wherein the biological sample is collected from a foodstuff or a mammal.

32. The method of claim 31, wherein the mammal is a human.

33. The method of claim 21, further comprising a step of determining whether an amplification product was produced as a result of the nucleic acid amplification reaction.

34. The method of claim 21, wherein the step of obtaining comprises collecting the swab sample.

35. A method comprising steps of:
obtaining an environmental sample from a source, wherein the environmental sample comprises a microorganism and the microorganism comprises a nucleic acid;
optionally concentrating the environmental sample to produce a concentrated sample, wherein the microorganism is concentrated about 2-fold to about 125-fold in the concentrated sample as compared to the environmental sample;
contacting the environmental sample or concentrated sample with a nucleic acid amplification reagent in a reaction vessel, wherein the environmental sample or concentrated sample is directly contacted with the nucleic acid amplification reagent without any intervening steps; and
performing a nucleic acid amplification reaction on the nucleic acid from the microorganism in the environmental sample or concentrated sample, wherein the nucleic acid amplification reaction is completed within less than 1 day from when the environmental sample was originally collected from the source.

36. The method of claim 35, wherein the amplification reaction is completed within less than 12 hours, less than 10 hours, less than 8 hours, less than 6 hours, less than 4 hours, less than 2 hours, less than 1 hour, less than 45 minutes, less than 30 minutes, less than 15 minutes, less than 10 minutes, less than 5 minutes, or less than 1 minute from when the environmental sample was originally collected from the source.

37. A method comprising steps of:
(a) obtaining a first environmental sample from a source, wherein the environmental sample comprises a microorganism and the microorganism comprises a nucleic acid;
(b) optionally concentrating the environmental sample to produce a concentrated sample, wherein the microorganism is concentrated about 2-fold to about 125-fold in the concentrated sample as compared to the environmental sample;
(c) contacting the environmental sample or concentrated sample with a nucleic acid amplification reagent in a reaction vessel, wherein the environmental sample or concentrated sample is directly contacted with the nucleic acid amplification reagent without any intervening steps;
(d) performing a nucleic acid amplification reaction on the nucleic acid from the microorganism in the environmental sample or concentrated sample, wherein the nucleic acid amplification reaction is optionally completed within less than 1 day from when the environmental sample was originally collected from the source; and
repeating steps (a), (c), (d) and optionally (b) on a second environmental sample from the same source within less than one month.

38. The method of claim 37, wherein steps (a), (c), (d) and optionally (b) are repeated on a new environmental sample from the same source on a monthly basis.

39. The method of claim 37, wherein steps (a), (c), (d) and optionally (b) are repeated on a second environmental sample from the same source within less than one week.

40. The method of claim 39, wherein steps (a), (c), (d) and optionally (b) are repeated on a new environmental sample from the same source on a weekly basis.


SEQUENCE LISTING (3 sequences)

SEQ ID NO: 1 - DNA, 22 bp, Artificial Sequence
  Forward Primer
ttgtcttatagcattggtgccg

SEQ ID NO: 2 - DNA, 21 bp, Artificial Sequence
  Reverse Primer
ccaattgagcgccactcatag

SEQ ID NO: 3 - DNA, 20 bp, Artificial Sequence
  Probe
  misc_feature (1)...(1): 5′ Cal Fluor610 labelled
  misc_feature (20)...(20): 3′ BHQ-2 labelled
caattgagcgccactcatag